SEQUENCE LISTING (2 sequences)

SEQ ID NO: 1 - DNA, 18 bp, Homo sapiens
caggcacgcaacttagtc

SEQ ID NO: 2 - DNA, 18 bp, Homo sapiens
gtaaggaggagggagcag

(19) United States
(12) Patent Application Publication
(10) Pub. No.: US 2008/0274121 A1
(43) Pub. Date: Nov. 6, 2008

(54) Inhibition of Angiogenesis by Mithramycin

(76) Inventors: James C. Yao, Houston, TX (US); Keping Xie, Houston, TX (US)

Correspondence Address: WILLIAMS, MORGAN & AMERSON, 10333 RICHMOND, SUITE 1100, HOUSTON, TX, 77042, US

(21) Appl. No.: 12/112,610

(22) Filed: Apr. 30, 2008

Related U.S. Application Data
(60) Provisional application No. 60/926,838, filed on Apr. 30, 2007.

(51) Int. Cl. A61K 39/395; A61K 31/704

(52) U.S. Cl. 424/158.1; 514/33

(57) ABSTRACT

We disclose a method of inhibiting angiogenesis in a mammal by administering by a subcutaneous or intraperitoneal route to a tissue in the mammal where angiogenesis is occurring a composition comprising mithramycin and a pharmaceutically-acceptable carrier. We have found a subcutaneous or intraperitoneal dose from about 10 μg mithramycin per kg body weight per day to about 500 μg mithramycin per kg body weight per day can be effective for inhibition of angiogenesis.

This application claims priority from U.S. provisional patent application Ser. No. 60/926,838, filed on Apr. 30, 2007, which is incorporated herein by reference.

The United States government may own rights in the present invention pursuant to grant numbers IP20—CA101936-01-PP4 and IR01—CA093829 from the National Cancer Institute, National Institutes of Health.

### BACKGROUND OF THE INVENTION

The present invention relates generally to the field of drug-based medical treatment. More particularly, it concerns the use of mithramycin in the treatment of angiogenesis.

Angiogenesis is the growth of new blood vessels, and naturally occurs in the body, in both health and disease. Angiogenesis occurs in the healthy body for healing wounds and for restoring blood flow to tissues after injury or insult. In females, angiogenesis also occurs during the monthly reproductive cycle (to rebuild the uterine lining and to mature the egg during ovulation) and during pregnancy (to build the placenta).

The healthy body controls angiogenesis through a series of “on” and “off” switches. The main “on” switches are known as angiogenesis-stimulating growth factors (“stimulators”). The main “off switches” are known as angiogenesis inhibitors (“inhibitors”). In general, angiogenesis is “turned off” by increased production of inhibitors relative to stimulators, although decreased production of stimulators relative to inhibitors is also possible.

When angiogenic growth factors are present locally or systemically in excess of angiogenesis inhibitors, the balance is tipped in favor of blood vessel growth.

Known angiogenesis stimulators include, but are not limited to, angiogenin, angiopoietin-1, Del-1, fibroblast growth factors: acidic (aFGF) and basic (bFGF), follistatin, granulocyte colony-stimulating factor (G-CSF), hepatocyte growth factor (HGF)/scatter factor (SF), interleukin-8 (IL-8), leptin, midkine, placental growth factor, platelet-derived endothelial cell growth factor (PD-ECGF), platelet-derived growth factor-BB (PDGF-BB), pleiotrophin (PTN), progranulin, proliferin, transforming growth factor-alpha (TGF-alpha), transforming growth factor-beta (TGF-beta), tumor necrosis factor-alpha (TNF-alpha), and vascular endothelial growth factor (VEGF)/vascular permeability factor (VPF), among others.

Known angiogenesis inhibitors include, but are not limited to, angioarrestin, angiostatin (plasminogen fragment), antiangiogenic antithrombin III, cartilage-derived inhibitor (CDI), CD59 complement fragment, endostatin (collagen XVIII fragment), fibronectin fragment, Gro-beta, heparinases, heparin hexasaccharide fragment, human chorionic gonadotropin (hCG), interferon alpha/beta/gamma, interferon inducible protein (IP-10), interleukin-12, Kringle 5 (plasminogen fragment), metalloproteinase inhibitors (TIMPs), 2-methoxyestradiol, placental ribonuclease inhibitor, plasminogen activator inhibitor, platelet factor-4 (PF4), prolactin 16 kD fragment, proliferin-related protein (PRP), retinoids, tetrahydrocortisol-S, thrombospondin-1 (TSP-1), transforming growth factor-beta (TGF-β), vasculostatin, and vasostatin (calreticulin fragment), among others.

Many of these stimulators and inhibitors of angiogenesis bind to specific cellular receptors to initiate signal transduction. Normal vascular biology depends on a balance between pro-angiogenic and anti-angiogenic forces. In many serious diseases states, the body loses control over angiogenesis. Angiogenesis-dependent diseases or diseases in which angiogenesis is a symptom result when new blood vessels grow excessively.

Excessive angiogenesis occurs in diseases such as cancer, diabetic blindness, age-related macular degeneration, rheumatoid arthritis, and psoriasis, among others. In these diseases, new blood vessels can do one or more of feed diseased tissues, destroy normal tissues, and in the case of cancer, allow tumor cells to escape into the circulation (tumor metastasis). Excessive angiogenesis often occurs when diseased cells produce abnormal amounts of angiogenic growth factors, overwhelming the effects of natural angiogenesis inhibitors.

A mechanism by which excessive angiogenesis can occur is the following. (1) Diseased or injured tissues produce and release angiogenic growth factors (proteins) that diffuse into nearby tissues. (2) The angiogenic growth factors bind to specific receptors located on the endothelial cells (EC) of nearby preexisting blood vessels. (3) Once growth factors bind to their receptors, the endothelial cells become activated. Signals are sent from the cell's surface to the nucleus. The endothelial cell's machinery begins to produce new molecules. (4) New molecules from the endothelial cell form pores in the sheath-like covering (basement membrane) surrounding existing blood vessels. (5) The endothelial cells proliferate and migrate out through the pores of the existing vessel towards the diseased tissue, sprouting a new blood vessel. (6) Adhesion molecules, also known as integrins (e.g., avb3, avb5), pull the new blood vessel sprout forward. (7) Additional molecules (matrix metalloproteinases, or MMP) are produced to dissolve the tissue in front of the sprouting vessel tip in order to accommodate it. As the vessel extends, the tissue is remolded around the vessel. (8) Sprouting endothelial cells roll up to form a blood vessel tube. (9) Individual blood vessel tubes connect to form blood vessel loops that can circulate blood. (10) Finally, newly formed blood vessel tubes are stabilized by specialized muscle cells (smooth muscle cells, pericytes) that provide structural support. Blood flow then begins.

A large body of scientific literature has shown that at least some proteins important in the regulation of angiogenesis, such as vascular endothelial growth factor (VEGF), are under transcriptional regulation by transcription factor Sp1. Sp1 is a zinc finger transcription factor that is important to the transcription of many cellular and viral genes that contain GC boxes in their promoters. Researchers have cloned additional transcription factors similar to Sp1 in their structural and transcriptional properties (Sp2, Sp3, and Sp4), thus defining the Sp1 multigene family. Such GC boxes have been described in the promoter of the VEGF, a key downstream regulator of angiogenesis. The promoter region for Sp1 contains binding sites for Sp1, creating an autocrine loop for amplification of Sp1 signaling. Although Sp1 has been perceived to be a basal transcription factor since its discovery, increasing evidence suggests that it regulates a variety of biological functions, including cell survival, growth, and differentiation and tumor development and progression. We previously reported that Sp1 overexpression is directly correlated with the angiogenic potential of and poor prognosis for human gastric and pancreatic cancer.

A number of agents have been reported for the modulation of angiogenesis (by which is meant either stimulation of angiogenesis, inhibition of angiogenesis, or both). A prescription gel called Regranex (recombinant human platelet-derived growth factor-BB, Ortho-McNeil Pharmaceuticals) has received FDA approval in treating diabetic foot ulcers. While complete regression of cancerous tumors has been shown following repeated cycles of antiangiogenic therapy using angiostatin and endostatin in animal models, their activity in human studies has been more limited. The monoclonal antibody drug Avastin (Bevacizumab) has been shown in large-scale clinical trials to inhibit tumor blood vessel growth and is presumed to be able to prolong survival in cancer patients.

However, resistance to existing anti-angiogenic agents does eventually develop. Further, modulators of angiogenesis have not been found for treatment of many diseases in which excessive angiogenesis occurs. A need remains for effective inhibitors of angiogenesis.

Mithramycin has previously been approved by FDA for human use (treatment of malignancy-associated hypercalcemia and testicular cancer). The initial investigational new drug (IND) filing was on Apr. 29, 1968 by Pfizer Labs. The initial FDA approval was granted on May 5, 1970. Mithramycin is a yellow crystalline compound which is produced by a microorganism, Streptomyces plicatus, although recombinant DNA and chemical synthesis techniques can also be used for its production. Mithramycin has fallen out of favor since other improved methods for controlling hypercalcemia have been developed. Extensive safety, toxicology, pharmacokinetics, tissue distribution and animal data already exist in the literature.

Mithramycin is an antibiotic that binds specific regions of DNA that are rich in guanine and cytosine. Mithramycin has been described to bind to consensus sequences in the Sp1 binding sites and preventing subsequent Sp1 binding. Thus, mithramycin may inhibit angiogenesis in several ways. First, it can bind to the GC boxes in the promoter sequences of key genes that regulate angiogenesis and inhibit Sp1 stimulated transcription of genes such as VEGF/VEGFR, bFGF/bFGFR, EGF/EGFR, IGF/IGFR, PDGF and others. Further, it can inhibit the binding of Sp1 to GC boxes in promoter sequence of Sp1 itself, and thereby inhibiting the autocrine amplification loop for Sp1 signaling (FIG. 1).

However, although mithramycin has previously been asserted to be effective for the treatment of malignancy-associated hypercalcimia and have direct cytotoxicity against cancer cells, mithramycin has not been previously been asserted to have anti-angiogenic properties.

### SUMMARY OF THE INVENTION

In one embodiment, the present invention relates to a method of inhibiting angiogenesis in a mammal by administering by a subcutaneous or intraperitoneal route to a tissue in the mammal where angiogenesis is occurring a composition comprising mithramycin and a pharmaceutically-acceptable carrier.

In mouse xenograft models of human cancer, we have found a subcutaneous or intraperitoneal dose from about 10 μg mithramycin per kg body weight per day to about 500 μg mithramycin per kg body weight per day can be effective for inhibition of angiogenesis.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. A, schematic structures of the minimal Sp1 promoters. The nucleotide positions and sequences of Sp1-binding sites and polymerase chain reaction forward and reverse primers flanking those sites for ChIP assay are shown. B, Sp1 and VEGF promoter reporter constructs were transfected into PANC-1 cells in triplicate and incubated for 12 hours. The cells were then incubated for another 24 hours in a medium alone or a medium containing 100 mg/ml BVZ or 0.1 mM MIT. Total protein lysates were harvested from the cell cultures for measurement of Sp1 and VEGF promoter activity using a luciferase assay kit. The relative Sp1 and VEGF promoter activities were assessed, and the activity in treated groups was expressed as the fold change of that in their respective control groups. C and D, chromatin was extracted from (C) PANC-1 cells that were incubated in vitro for 2 days in a medium alone or a medium containing 100 mg/ml BVZ or 0.1 mM MIT and (D) tumors formed by PANC-1 cells in nude mice that received treatment as described FIG. 2A. The ChIP assay was performed using a specific anti-Sp1 antibody and oligonucleotides flanking the respective Sp1 and VEGF promoter regions containing Sp1-binding sites (lane 1, input chromatin DNA; lane 2, chromatin DNA with control Ig G; and lane 3, chromatin DNA with anti-Sp1 antibody). Ctr, control. This was one representative experiment of two with similar results.

FIG. 2. Western blot analysis of Sp1 expression in carcinoid cancer cell line H727, as described in Example 1.

FIG. 3. The inhibition of SP1 by mithramycin A in H727 tumor (A) is associated with significant decrease in expression of PDGF, VEGF, and EGRF (B).

FIG. 4. Tumor weight and ascites volume of SKOV3ip1 tumors under various treatment regimens, as described in Example 3.

FIG. 5. BxPC-3 cells were injected into the subcutis of groups of mice (n=3). When tumors reached around 4 mm in diameter, animals received i.p. injection of PBS or 0.20 mg/kg MIT. Tumors were harvested 6 to 48 hours after MIT injection. Total protein lysates were extracted for analysis of SP1 expression using Western blotting.

FIG. 6. BxPC-3 cells were injected into the subcutis of groups of mice (n=5). When tumors reached 4 mm in diameter, animals received injections of PBS (controls), MIT (0.10, 0.20, or 0.40 mg/kg (s.c. or i.p.) twice a week. Tumors were measured once every week, and at each measurement, the mean±SD tumor volume in the five mice in each group was calculated. Note that s.c. injection of MIT was found to be equally, if not more, effective in suppressing the growth of BxPC-3.

FIG. 7. H727 carcinoid tumor volume during treatment course in nude mouse xenograft model for various doses and administration routes of mithramycin.

FIG. 8. Suppression of angiogenesis as manifested in representative mice by decreased microvessel density in H727 carcinoid tumor growing in mice receiving PBS or mithramycin.

FIG. 9. Western blot analysis of Sp1 expression in pancreatic cancer cell lines, as described in Example 1.

FIG. 10. Dose-dependent antitumor effects of BVZ and MIT in xenograft models of human pancreatic cancer. A and B, BxPC3 cells were injected into the subcutis of nude mice (n=5). When tumors reached around 4 mm in diameter, the animals received different doses of (A) BVZ (0.10 [B-100], 0.25 [B-25], and 0.50 [B-50] mg/kg) and (B) MIT (0.10 [M-10], 0.20 [M-20], and 0.40 [M-40] mg/kg) via intraperitoneal injection twice a week. Tumors were measured once every week, and at each measurement, the mean±standard deviation (SD) tumor volume in the five mice in each group was calculated. C and D, the control mice and mice that received (C) BVZ and (D) MIT were weighed at the time of experiment termination, and the weights were expressed as the mean±SD. The asterisks indicate the statistical significance (P<0.01) in a comparison between the treated and respective control groups. This was one representative experiment of two with similar results.

FIG. 11. Synergistic antitumor effects of treatment with BVZ and MIT in xenograft models of human pancreatic cancer. Both (A) BxPC3 and (B) PANC-1 cells were injected into the subcutis of groups of mice (n=5). Specifically, animals received injections of PBS (controls), BVZ (0.025 mg), MIT (0.010 mg/kg), or B+M. Tumors were measured once every week, and at each measurement, the mean±SD tumor volume in the five mice in each group was calculated. The asterisks in A and B indicate the statistical significance (P<0.01) in a comparison between the treated and respective control groups. C, representative tumor sizes in each group of BxPC3 model mice. This was one representative experiment of two with similar results.

FIG. 12. Prolonged survival of mice that received treatment with BVZ and MIT in xenograft models of human pancreatic cancer. PANC-1 cells were injected into the pancreas of nude mice (17 to 20 mice/group). The mice received treatment as described in FIG. 2. The entire experiment was terminated 120 days after tumor-cell injection. A, animal survival was monitored daily until termination of the experiment. Cum, cumulative. B, tumor growth in the pancreas and metastasis in the liver and/or other organs were evaluated and expressed as the incidence (%). The asterisks indicate the statistical significance (P<0.01) in a comparison between the treated and respective control groups. This was one representative experiment of two with similar results.

FIG. 13. Analysis of gene expression and microvessel formation in tumor tissues. The tumor tissues described in FIG. 2 were collected and processed as described in Materials and Methods. A, total protein lysates were harvested from tumor tissues, and the level of protein expression was determined using Western blot analysis. Equal protein-sample loading was monitored by probing the same membrane filter with an anti-β-actin antibody. NS, nonspecific. B, the levels of Sp1 and VEGF expression were quantitated and expressed as fold change. C, MVD was quantitated according to CD31 staining. D, immunohistochemical staining was performed using specific antibodies against Sp1, VEGF, and CD31. Representative photos of the stains are shown.

FIG. 14. Effects of treatment with BVZ and MIT on the growth of and gene expression in human pancreatic cancer cells in vitro. (A) BxPC3 and (B) PANC-1 cells were incubated for 1 to 5 days in a medium alone or a medium containing 100 μg/ml BVZ. The viable cells were counted every 24 hours. C, PANC-1 cells were incubated for 6 to 48 hours in a medium alone or a medium containing 100 μg/ml BVZ. Total protein lysates were harvested from the cell cultures, and the level of Sp1, EGFR, and VEGF protein expression was determined using Western blot analysis. Equal protein-sample loading was monitored by probing the same membrane filter with an anti-glyceraldehyde-3-phosphate dehydrogenase antibody (GAPDH). D and E, PANC-1 cells were treated with MIT at concentrations ranging from 0.1 to 1.2 μM for 24 and 48 hours. (D) Cytotoxicity was assessed using the MTT assay, and (E) the level of gene expression was determined using Western blot analysis. NS, nonspecific. This was one representative experiment of two with similar results.

FIG. 15. Effects of treatment with BVZ and MIT on Sp1 recruitment into the Sp1 promoter in human pancreatic cancer cells in vitro and in vivo. A, schematic structures of the minimal Sp1 promoters. The nucleotide positions and sequences of Sp1-binding sites and polymerase chain reaction forward and reverse primers flanking those sites for ChIP assay are shown. B, Sp1 and VEGF promoter reporter constructs were transfected into PANC-1 cells in triplicate and incubated for 12 hours. The cells were then incubated for another 24 hours in a medium alone or a medium containing 100 μg/ml BVZ or 0.1 μM MIT. Total protein lysates were harvested from the cell cultures for measurement of Sp1 and VEGF promoter activity using a luciferase assay kit. The relative Sp1 and VEGF promoter activities were assessed, and the activity in treated groups was expressed as the fold change of that in their respective control groups. C and D, chromatin was extracted from (C) PANC-1 cells that were incubated in vitro for 2 days in a medium alone or a medium containing 100 μg/ml BVZ or 0.1 μM MIT and (D) tumors formed by PANC-1 cells in nude mice that received treatment as described in FIG. 2A. The ChIP assay was performed using a specific anti-Sp1 antibody and oligonucleotides flanking the respective Sp1 and VEGF promoter regions containing Sp1-binding sites (lane 1, input chromatin DNA; lane 2, chromatin DNA with control Ig G; and lane 3, chromatin DNA with anti-Sp1 antibody). Ctr, control. This was one representative experiment of two with similar results.

FIG. 16. Inhibition of expression of Sp1 and its downstream molecules by MIT in human carcinoid cells. H727 cells were incubated in medium or medium containing 0.36 μM MIT for 24 hours. mRNA was extracted for Northern blot analysis of Sp1 expression (A1). H727 and BON-1 cells were incubated in medium or medium containing 0-0.36 μM MIT for 1 to 24 hours. Total protein lysates were extracted for Western blot analyses of expression of Sp1 and its downstream molecules, including VEGF, EGFR and PDGFA (A2). B. Chromatin was prepared from the cells and ChIP assay was performed as described in Materials and Methods. Note that MIT inhibits Sp1 expression and its downstream molecules. The asterisks indicate the statistical significance (P<0.01) in a comparison between the treated and respective control groups. This was one representative experiment of two with similar results.

FIG. 17. Inhibition of human carcinoid angiogenesis by MIT in animal models. H727 and BON-1 cells were injected into the subcutis of nude mice (n=5). When tumors reached around 4 mm in diameter, the animals received different doses of MIT (0.10 [M-10], 0.20 [M-20], and 0.40 [M-40] mg/kg) via intraperitoneal injection twice a week. Tumors were measured once every week, and at each measurement, the mean±standard deviation (SD) tumor volume in the five mice in each group was calculated (A). The residual H727 tumors were harvested and protein expression of Sp1 and VEGF, EGFR and PDGFA was determined by Western blot analyses (B). CD31 staining of residual H727 from mice treated with PBS or 0.20 MIT mg/kg was performed using frozen sections and MVD was assessed by counting (C). This was one representative experiment of two with similar results.

FIG. 18. Effects of treatment with BVZ and MIT on the growth of and gene expression in human carcinoid cells in vitro. A. H727 and BON-1 cells were incubated for 5 days in a medium alone or a medium containing 25 to 100 μg/ml BVZ. The viable cells were counted. B. H727 cells were incubated for 5 days in a medium alone or a medium containing 100 μg/ml BVZ. Total protein lysates were harvested from the cell cultures, and the level of Sp1 and VEGF protein expression was determined using Western blot analysis. Equal protein-sample loading was monitored by probing the same membrane filter with an anti-GAPDH. C. H727 and BON-1 cells were treated with MIT at concentrations ranging from 0.1 to 2.5 μM for 24 hours. Cytotoxicity was assessed using the MTT assay. This was one representative experiment of two with similar results.

FIG. 19. Synergistic antitumor effects of BVZ and MIT in human carcinoid model. H727 and BON-1 cells were injected subcutaneously into nude mice. When their tumors reached 4 mm in diameter, we gave the animals different doses of BVZ (25 μg) alone or in combination with MIT (0.1 mg/kg, with slight antitumor effects) via intraperitoneal injection twice a week. A. Tumors were measured once every week, and at each measurement, the mean±SD tumor volume in the five mice in each group was calculated. B. Mouse body weights were measured. The asterisks indicate the statistical significance (P<0.01) in a comparison between the treated and respective control groups. This was one representative experiment of two with similar results.

FIG. 20. Upregulation of Sp1 and VEGF expression by treatment with BVZ and its reversal by treatment with MIT. The tumor tissues described in FIG. 4 were collected and processed as described in Materials and Methods. A. Frozen sections made from the tumors were used CD31 staining and MVD assessment. Representative photos of the stains are shown. B. Total protein lysates were harvested from tumor tissues, and the expression levels of Sp1, VEGF, EGFR and PDGFA protein were determined using Western blot analyses. Equal protein-sample loading was monitored by probing the same membrane filter with an anti-GAPDH antibody. C. The expression levels of Sp1, VEGF, EGFR and PDGFA were further determined by calculating the ratios between those genes and GAPDH. This was one representative experiment of two with similar results.

### DESCRIPTION OF ILLUSTRATIVE EMBODIMENTS

In one embodiment, the present invention relates to a method of inhibiting angiogenesis in a mammal by administering by a subcutaneous or intraperitoneal route to a tissue in the mammal where angiogenesis is occurring a composition comprising mithramycin and a pharmaceutically-acceptable carrier.

Any mammal can be the subject of the method. In one embodiment, the mammal is Homo sapiens or a mammal having economic or esthetic utility, such as horses, cattle, sheep, dogs, or cats. In a further embodiment, the mammal is Homo sapiens.

Any tissue to which the composition can be provided by subcutaneous or intraperitoneal delivery, and in which angiogenesis is occurring, can be the target of the administering step. The tissue can be the subcutis of the skin or a tissue of the abdominal cavity or an organ therein, but need not be. Subcutaneous or intraperitoneal administration can be used for systemic delivery of the composition. In one embodiment, the tissue is a component of the stomach, the small intestine, the large intestine, the pancreas, the ovaries, the eyes, the skin, or one or more joints.

The occurrence of angiogenesis can be a symptom of a medical condition, for which at least partial relief can be effected by the inhibition of angiogenesis. In one embodiment, the mammal suffers from a medical condition selected from the group consisting of gastric cancer, carcinoid cancer, pancreatic cancer, and ovarian cancer. In another embodiment, the mammal suffers from a medical condition is selected from the group consisting of macular degeneration, diabetic neuropathy, psoriasis, and rheumatoid arthritis.

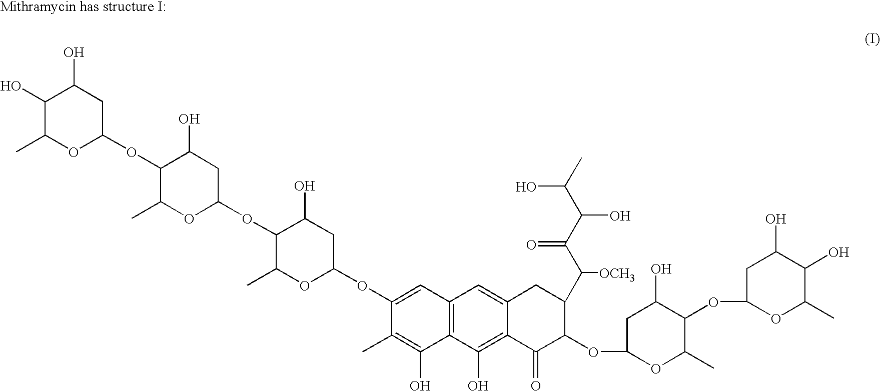

The pharmaceutically-acceptable carrier is an aqueous solution, such as water, normal saline, or a buffered aqueous solution, known in the art for delivery of water-soluble drugs. In one embodiment, the pharmaceutically-acceptable carrier is selected from the group consisting of distilled water and phosphate-buffered saline (PBS).

The composition containing mithramycin and the pharmaceutically-acceptable carrier can be prepared by any techniques known in the art. As a general rule, the composition can be sterilized by any technique known in the art for the sterilization of aqueous mithramycin solutions. The concentration of mithramycin in the composition is not crucial. The concentration of mithramycin will generally be less than its maximum possible concentration in aqueous solution. The concentration of mithramycin will generally be high enough that both the total volume of the composition will be low enough to be readily administered to the patient and an efficacious dose of mithramycin can be delivered. Appropriate doses are discussed below.

In one embodiment, administering delivers a dose from about 10 μg mithramycin per kg body weight per day to about 500 μg mithramycin per kg body weight per day.

In addition to the mithramycin and the pharmaceutically-acceptable carrier, the composition can further comprise one or more other materials, such as adjuvants, preservatives, local anesthetics (such as 1% lidocaine or bupivacaine, which may be appropriate for subcutaneous injection), or other ingredients active against angiogenesis or other medical conditions. In one embodiment, the composition further comprises a VEGF antagonist or a PDGF antagonist. In a further embodiment, the VEGF antagonist is bevcaizumab or the PDGF antagonist is imatinib.

The concentration of the other material or materials in the composition can be chosen using the same considerations discussed above and others apparent to the skilled artisan. In an embodiment wherein the VEGF antagonist is bevcaizumab, administering delivers a dose from about 100 μg bevcaizumab per kg body weight per day to about 4 mg bevcaizumab per kg body weight per day. In human applications, doses of 1 mg/kg to 15 mg/kg given weekly, biweekly, or triweekly, as convenient, may be used. In another embodiment, doses of 1.25 mg/kg to 7.5 mg/kg given weekly, biweekly, or triweekly, as convenient, may be used.

We have found that mithramycin can regulate the transcription of multiple angiogenic molecules. For this reason, it can be used in the therapy of diseases where aberrant angiogenesis plays a role.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

Summary: We performed experiments to optimize use of mithramycin for antiangiogenesis. In vivo studies showed that anti-angiogenic activity is achieved at significantly lower (up to 10-fold lower) doses then were used in the art. Anti-angiogenic activity may require a more frequent dosing regimen, such as weekly or twice a week, than the monthly dosing used in previously approved cancer treatment. In addition to the intravenous route of administration known in the art, we found that mithramycin also inhibits angiogensis when given via subcutaneous or intraperitoneal routes.

Mithramycin's anti-angiogenic activity was explored in several ways. In xenograft models of human cancer, we demonstrate below single agent anti-tumor activity in a variety of models (H727 carcinoid, SKOV3 ovarian cancer, and BxPC-3 pancreatic cancer). In addition, additive or synergistic activity was demonstrated when combined with agents targeting VEGF or platelet-derived growth factor (PDGF).

Material and Methods

Cell lines: The human bronchial carcinoid cancer cell line NCI-H727 (ATCC: CRL-5815) was purchased from the American Type Culture Collection (ATCC, Manassas, Va.). Human pancreatic carcinoid cell line BON-1 was provided by Dr. Kjell Oberg (Uppsala University, Uppsala, Sweden). AsPC-1, BxPC3, FG, and PANC-1 human pancreatic adenocarcinoma cell lines were purchased from the American Type Culture Collection (Manassas, Va.). The cell lines were maintained in plastic flasks as adherent monolayers in minimal essential medium supplemented with 10% fetal bovine serum, sodium pyruvate, nonessential amino acids, L-glutamine, and a vitamin solution (Flow Laboratories, Rockville, Md.).

Mithramycin A was purchased from Sigma Aldrich Co. Cat#M-7393 (1 mg/vial)

Adherent monolayer cultures were maintained in 75 cm² flasks (Falcon) and incubated under the conditions of a humid atmosphere with 5% CO₂ at 37° C. The culture medium was changed every 2 to 3 days, and subculture monolayer cells almost once a week, with a subcultivation ratio of 1:4. The cells in log-phase growth were used for the following study.

Female BLAB/c nu/nu nude mice (NCl), at 6-7 weeks of age, weight about 25 g, were purchased from the Animal Production Area of the National Cancer Institute Frederick Cancer Research and Development Center (Frederick, Md.). The mice were housed and maintained in a laminar airflow cabinet under specific pathogen-free conditions, and were housed for at least 7 days before use. All facilities used in this protocol were approved by the Animal Care and Use Committee of M.D. Anderson Cancer Center, Houston, Tex.

Subcutaneous Implantation of Tumor Cells: The right flank of each mouse was prepared sterilely and then given subcutaneous injection of 1.5*10⁷ cells/mouse in 0.15 ml serum-free medium. The date of tumor cell implantation was marked as d0. The tumors were allowed to grow for 5 days. Food and drinking water were available.

Animal Groups and Drug Delivery: After 5 days, nude mice implanted with cell lines derived from various human cancers. Implanted animals were randomized and classified into different treatment groups as follows (5 mice/group). Studied drugs were intraperitoneally injected with mithramycin and/or different targeted drugs twice per week, while the control group received only PBS. Treatments were delivered as intra-peritoneal injections of 0.05 mL total volume using a 27-gauge needle.

Tumor Size Evaluation: Measure the tumor size at each timepoint for drug administration until the mouse is sacrificed. Tumor size was measured in two dimensions using calipers, and the changes in tumor surface area (product of the two diameters) compared to the baseline tumor size on the first day of antibody injection was determined. Percent change from baseline was calculated by comparing the baseline value to the tumor size on each time of measurement. The tumor size may be calculated according to the following formula: V=πa²b/6(a: maximal tumor diameter; b: minimal tumor diameter).

Animal sacrifice and specimen collection: The mice were killed when moribund or 2 weeks after treatment. The body weights were recorded. The size and weight of the primary subcutaneous tumors and the incidence of liver and lung metastases were counted and recorded. Histopathologic studies confirmed the nature of the disease.

### Example 1

Mithramycin Activity in Human Cancer Cell Lines

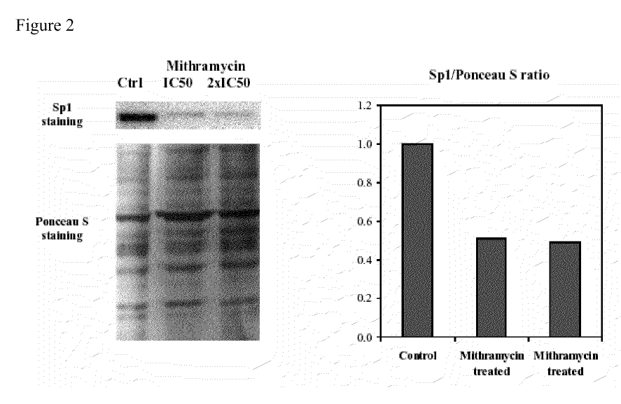

H727 carcinoid cancer cell lines were cultured in 10% FCS medium overnight, then treated with IC50 and 2*IC50 concentration of Mithramycin for 24 hours. Sp1 were determined by Western blot analysis with antibodies specific for Sp1, as shown in FIG. 2. Equal loading was determined by Ponceau S staining after membrane transfer.

FIG. 9 shows a similar experiment using pancreatic cancer cell lines PANC-1 and Pau-8902.

### Example 2

### Mithramycin Activity Against Human Cancers in Nude Mouse Xenograft Models

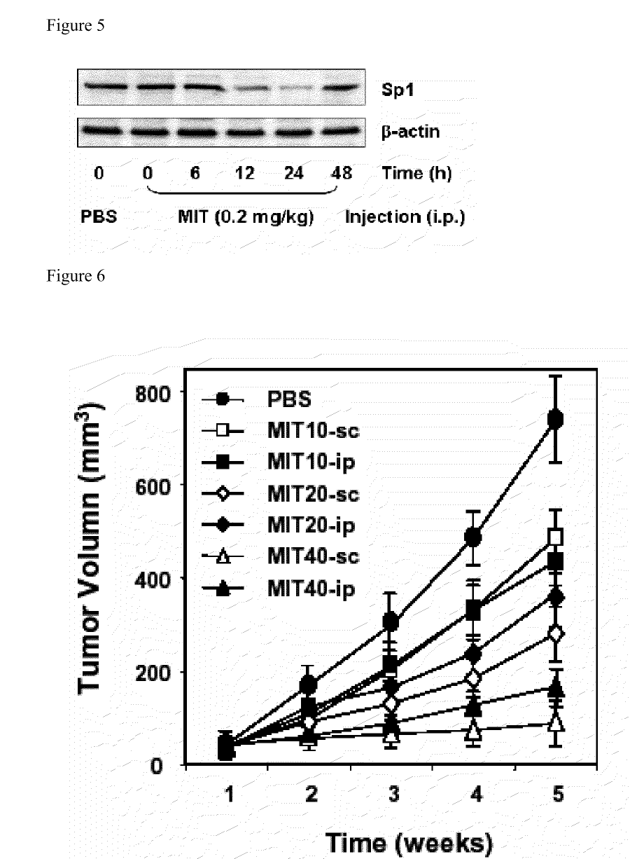

The activity of mithramycin was studied in a variety of human cancer xenograft models including carcinoid tumors, pancreatic cancer, and ovarian cancer. In a series of studies, we evaluated the dose of mithramycin required for Sp1 inhibition as well as the duration of Sp1 inhibition following administration of mithramycin. We found that the dose of mithramycin required for inhibition of Sp1 in nude mouse human cancer xenograft models to be significantly lower then the dose used for other indications in mice experiments. The duration of Sp1 inhibition is 24 hours (FIG. 5).

Our data suggest that the previously used MTD based method for dosing mithramycin in the clinics is not optimal for anti-angiogenic activity. Mithramycin at significantly lower doses given weekly or twice weekly can inhibit Sp1 in a continuous manner with lower toxicity. Further, in our experiments mithramycin had significant activity when given either intraperitoneally or subcutaneously (FIG. 6) suggesting that alternative dosing methods could be used in future clinical studies.

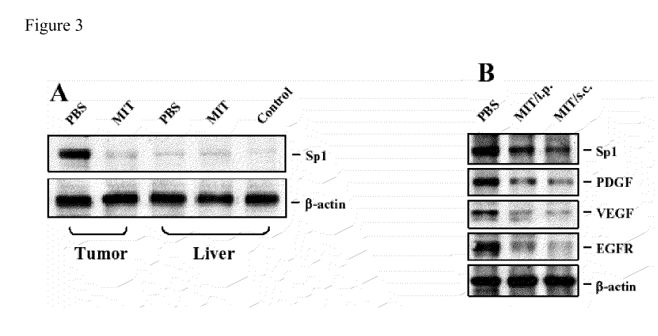

FIG. 3 shows the inhibition of Sp1 by mithramycin A is associated with significant decrease in expression of PDGF, VEGF, and EGFR.

Further, synergistic anti-tumor activity was observed when mithramycin was combined with other agents targeting angiogenesis such as VEGF inhibitor bevcaizumab. Details of experiments in pancreatic cancer, ovarian cancer, and carcinoid tumors are attached or outlined below.

### Example 3

### Suppression of SKOV3 ip1 Human Ovarian Cancer Growth by MIT and BVZ in Nude Mouse Xenograft Models

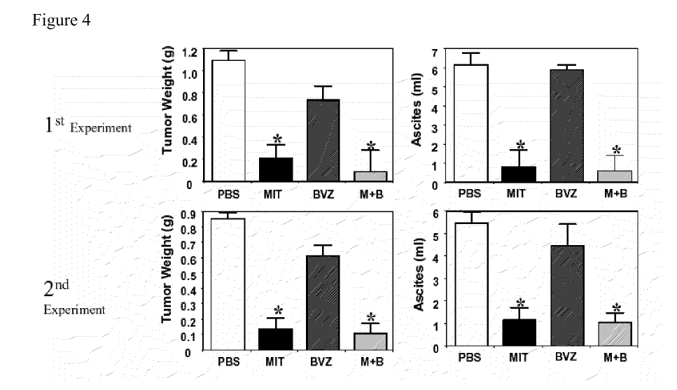

SKOV31p1 cells were injected into the peritoneal cavity of groups of mice (n=5). When tumors reached 4 mm in diameter, animals received injections of PBS (controls), BVZ (0.025 mg), MIT (0.10 mg/kg), or BVZ+MIT twice a week. The entire experiment was terminated 30 days after tumor-cell injection. Tumor weight and ascites were measured, as shown in FIG. 4. Note that MIT treatment alone was highly effective in controlling ovarian cancer growth.

### Example 4

### Suppression of H727 Carcinoid Cell Growth by MIT in Nude Mouse Xenograft Models

H727 carcinoid cells were injected into the subcutis of groups of mice (n=5). When tumors reached 4 mm in diameter, animals received injections of PBS (controls), MIT (0.10, 0.20, or 0.40 mg/kg (s.c. or i.p.) twice a week. Tumors were measured once every week, and at each measurement, the mean±SD tumor volume in the five mice in each group was calculated.

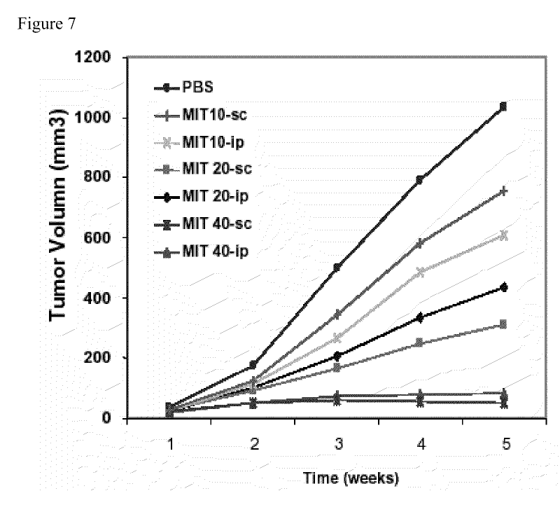

Note that s.c. injection of MIT was found to be equally, if not more, effective than i.p. injection in suppressing the growth of H727, as shown in FIG. 7.

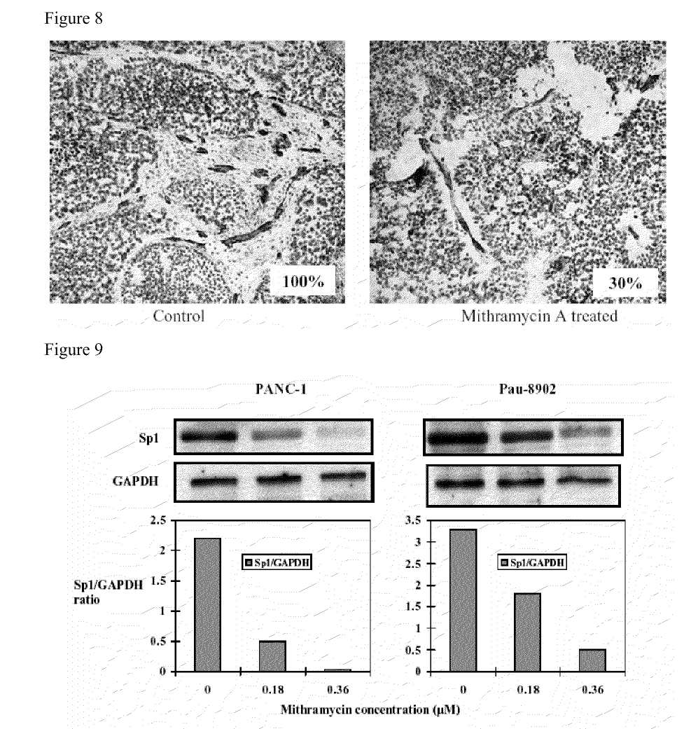

FIG. 8 shows suppression of angiogenesis as manifested in representative mice by decreased microvessel density in H727 carcinoid tumor growing in mice receiving PBS or mithramycin via i.p. or s.c. injection.

### Example 5

### Treatment with Bevacizumab Upregulates Expression of the Transcription Factor Sp1 and its Downstream Target Genes in Human Pancreatic Cancer Cells

### Molecular Basis of the Synergistic Antiangiogenic Activity of Bevacizumab and Mithramycin A

Abstract

Previous studies have shown that human pancreatic adenocarcinoma cells overexpress proangiogenic factors such as vascular endothelial growth factor (VEGF) and that the transcription factor Sp1 plays a critical role in VEGF inducible and constitutive expression. However, the impact of antiangiogenic therapy on the Sp1 VEGF pathway remains unclear. Treatment with bevacizumab (BVZ), a neutralizing antibody against VEGF A, suppressed human pancreatic cancer growth in nude mice. Gene expression analyses revealed that this treatment substantially upregulated the expression of Sp1 and its downstream target genes, including VEGF and epidermal growth factor receptor, in tumor tissues, whereas it did not have this effect on pancreatic cancer cells in culture. Treatment with mithramycin A, a Sp1 inhibitor, suppressed the expression of Sp1 and its downstream target genes in both cell culture and tumors growing in nude mice. Combined treatment with BVZ and mithramycin A produced synergistic tumor suppression, which was consistent with suppression of the expression of Sp1 and its downstream target genes. Thus, treatment with BVZ may block VEGF function but activate the pathway of its expression via positive feedback. Given the fact that Sp1 is an important regulator of the expression of multiple angiogenic factors, BVZ-initiated upregulation of Sp1 and subsequent overexpression of its downstream target genes may profoundly affect the potential angiogenic phenotype and effectiveness of antiangiogenic strategies for human pancreatic cancer.

Introduction

Pancreatic cancer is currently the fourth leading cause of cancer-related deaths in the United States. The median survival duration from diagnosis to death is about 4 to 6 months, and the overall 5-year survival rate is less than 5% (1-3). A full understanding of the cellular and molecular mechanisms of the development and progression of pancreatic cancer is crucial for identifying new targets of effective treatment modalities for this deadly disease. Among the various potential targets are numerous proangiogenic and antiangiogenic factors released by tumor and host cells (4-6). These factors regulate angiogenesis, which determines the growth and metastasis of pancreatic cancer (6-8). Of the numerous angiogenic factors discovered thus far, studies have identified vascular endothelial growth factor (VEGF) as a key mediator of tumor angiogenesis (9-11). Authors have reported elevated expression of VEGF in human pancreatic cancer specimens (12, 13), that its expression level correlates with microvessel density (MVD) (4, 6, 14-16), and that VEGF-targeted therapy significantly inhibits angiogenesis and growth of pancreatic cancer in animal models (4, 6, 17). Other identified proangiogenic factors that are overexpressed in human pancreatic cancer include epidermal growth factor (EGF) and EGF receptor (EGFR) (4, 18), insulin-like growth factor (IGF)-T and IGF-I receptor (IGF-IR) (19), hepatocyte growth factor (HGF) and its receptor Met (20, 21), and fibroblast growth factor (FGF) and FGF receptor (22).

The molecular mechanisms by which these angiogenic molecules are regulated remain unclear. Our previous studies demonstrated that the transcription factor Sp1 plays an important role in regulating expression of VEGF and angiogenesis of human pancreatic cancer (23, 24). However, whether and, if so, how treatments targeting molecules such as VEGF impact the expression of Sp1 and its downstream target genes, including VEGF, are unclear. We hypothesize that strategies such as neutralization of VEGF by treatment with bevacizumab (BVZ) lead to feedback activation of Sp1 and subsequent upregulation of VEGF, EGFR, IGF-IR, and other factors, leading to BVZ resistance, whereas blockade of Sp1 expression and function sensitizes tumor to BVZ and/or reverses BVZ resistance.

Sp1 is a zinc finger transcription factor that is important to the transcription of many cellular and viral genes that contain GC boxes in their promoters. Researchers have cloned additional transcription factors similar to Sp1 in their structural and transcriptional properties (Sp2, Sp3, and Sp4), thus forming the Sp1 multigene family (25). Although Sp1 has been perceived to be a basal transcription factor since its discovery, increasing evidence suggests that it regulates a variety of biological functions, including cell survival, growth, and differentiation and tumor development and progression (25-29). We previously reported that Sp1 overexpression is directly correlated with the angiogenic potential of and poor prognosis for human gastric and pancreatic cancer (22, 24, 30). Therefore, Sp 1 inhibitors such as mithramycin A (MIT) may have profound antiangiogenic effects.

MIT, also known as aureolic acid and plicamycin (Mithracin), is an aureolic acid-type polyketide produced by various soil bacteria of the genus Streptomyces (31, 32). In the past, MIT has been used to manage hypercalcemia in patients bone metastases from various malignancies, while some have also used it to treat Paget's disease and several types of cancer, including testicular carcinoma, chronic myeloid leukemia, and acute myeloid leukemia (33-35). Furthermore, researchers have shown that MIT acts as a neuroprotective drug (36). MIT binds to GC-rich regions in chromatin and interferes with the transcription of genes that bear GC-rich motifs in their promoters (33, 37). Its mechanism of action involves a reversible interaction with double-stranded DNA with GC-base specificity. MIT is believed to act, in part, by selectively regulating transcription of genes that have GC-rich promoter sequences (38). In addition, recent studies have shown that MIT sensitizes tumor cells to apoptosis induced by tumor necrosis factor and inhibits p53-mediated transcriptional responses (39, 40). Inhibition of Sp1 activity is considered to be a major mechanism of the antitumor activity of MIT. Thus, we performed the present study to determine the effect of and molecular basis for combined use of BVZ and MIT as antiangiogenic therapy for pancreatic cancer.

Materials and Methods

Chemicals and reagents. MIT (1 mg/vial crystal powder; lot 055K4011) was purchased from Sigma Chemical Co. (St. Louis, Mo.) and diluted in sterile water. BVZ (25 mg/ml; NDC 50242-060-01) from Genentech, Inc. (South San Francisco, Calif.) was purchased. For animal experiments, MIT (0.1 to 0.4 mg/kg body weight) and BVZ (25 to 100 μg/mouse) were administered by intraperitoneal injection twice a week or as indicated otherwise.

Cell lines and culture conditions. The human pancreatic adenocarcinoma cell lines BxPC3 and PANC-1 were purchased from the American Type Culture Collection (Manassas, Va.). The cell lines were maintained in plastic flasks as adherent monolayers in minimal essential medium supplemented with 10% fetal bovine serum, sodium pyruvate, nonessential amino acids, L-glutamine, and a vitamin solution (Flow Laboratories, Rockville, Md.).

Animals. Female athymic BALB/c nude mice were purchased from The Jackson Laboratory (Bar Harbor, Me.). The mice were housed in laminar flow cabinets under specific pathogen-free conditions and used when they were 8 weeks old. The animals were maintained in facilities approved by the Association for Assessment and Accreditation of Laboratory Animal Care in accordance with the current regulations and standards of the U.S. Department of Agriculture, U.S. Department of Health and Human Services, and National Institutes of Health.

Western blot analysis. Whole-cell lysates were prepared from human pancreatic cancer cell lines and tissues (23). Standard Western blotting was performed using polyclonal rabbit antibodies against human and mouse Sp1, VEGF, and EGFR (Santa Cruz Biotechnology, Santa Cruz, Calif.) and the anti-rabbit IgG antibody, a horseradish peroxidase-linked F(ab′)₂ fragment obtained from a donkey (Amersham, Arlington Heights, Ill.). Equal protein-sample loading was monitored by probing the same membrane filter with antibodies against anti-β-actin or glyceraldehyde-3-phosphate dehydrogenase (GAPDH) (23). The probe proteins were detected using the Amersham enhanced chemiluminescence system according to the manufacturer's instructions.

Immunohistochemical analysis and quantification of tumor MVD. For CD31 staining, frozen tissue sections (5 μm thick) were fixed in acetone. Endogenous peroxidase in the specimens was blocked using 3% hydrogen peroxide in phosphate-buffered saline (PBS) for 12 minutes. The specimens were incubated for 20 minutes at room temperature in a protein-blocking solution consisting of PBS (pH 7.5) containing 5% normal horse serum and 1% normal goat serum and then incubated overnight at 4° C. in a 1:100 dilution of monoclonal goat anti-CD31 (PECAM1-M20), polyclonal rabbit anti-Sp 1, or polyclonal rabbit anti-VEGF antibodies (Santa Cruz Biotechnology). The specimens were then rinsed and incubated with peroxidase-conjugated anti-goat or anti-rabbit IgG for 1 hour at room temperature. Next, the slides were rinsed with PBS and incubated with diaminobenzidine (Research Genetics, Huntsville, Ala.) for 5 minutes. Frozen sections of the specimens were then washed three times with distilled water, counterstained with Mayer's hematoxylin (Biogenex Laboratories, San Ramon, Calif.), and washed once each with distilled water and PBS. The slides were mounted with Universal Mount (Research Genetics) and examined under a bright-field microscope. A positive reaction was indicated by a reddish-brown precipitate in the cytoplasm (CD31) or nuclei (Sp1). For quantification of tumor MVD, vessels on each section were counted in five high-power fields (magnification, ×200 [×20 objective and ×10 ocular]) as described previously (41).

Sp1 promoter constructs and analysis of Sp1 promoter activity. The minimal Sp1 promoter reporters in pGL3 luciferase constructs were generated and used as described previously (23, 42). To examine the transcriptional regulation of the Sp1 promoters by BVZ and MIT, PANC-1 cells were seeded to about 80% confluence in six-well plates (in triplicate) and transiently transfected with 0.6 μg of minimum Sp1 reporter plasmids and 0.3 μg of effector expression plasmids as indicated in each experiment using Lipofectamine (Invitrogen, Carlsbad, Calif.) according to the manufacturer's instructions. The reporter luciferase activity was measured 48 hours later using a luciferase assay kit (Promega, Madison, Wis.). Promoter activity was normalized according to the protein concentration as described previously (23, 42).

Chromatin immunoprecipitation. Chromatin was prepared from cells and tumors as described previously (42). Chromatin immunoprecipitation (ChIP) assay was performed using the Chromatin Immunoprecipitation Assay Kit (Upstate Cell Signaling Solutions, Lake Placid, N.Y.) according to the manufacturer's instructions. Briefly, DNA cross-binding proteins were cross-linked with DNA and lysed in sodium dodecyl sulfate lysis buffer. The lysate was sonicated to shear DNA to 200 to 500 bp. After preclearing with a salmon sperm DNA/protein A agarose-50% slurry for 30 minutes at 4° C., chromatin samples were immunoprecipitated overnight with no antibody or an anti-Sp1 antibody (PEP2). The region between −224 and -53 bp of the Sp1 promoter was amplified using the following primers: sense, 5′-caggcacgcaacttagtc-3′ (SEQ ID NO: 1), and antisense, 5′-gtaaggaggagggagcag-3′ (SEQ ID NO:2). Polymerase chain reaction products were separated on a 1.5% agarose gel, stained with ethidium bromide, and visualized under ultraviolet light.

Statistical analysis. Each experiment was performed independently at least twice with similar results; one representative experiment is presented. The significance of the in vitro data was determined using Student's t-test (two-tailed), whereas the significance of the in vivo data was determined using the two-tailed Mann-Whitney U test. For the in vivo experiments, overall survival was calculated by the method of Kaplan and Meier. Log rank test was used to compare the survival duration between groups. P≦0.05 was deemed significant.

Results

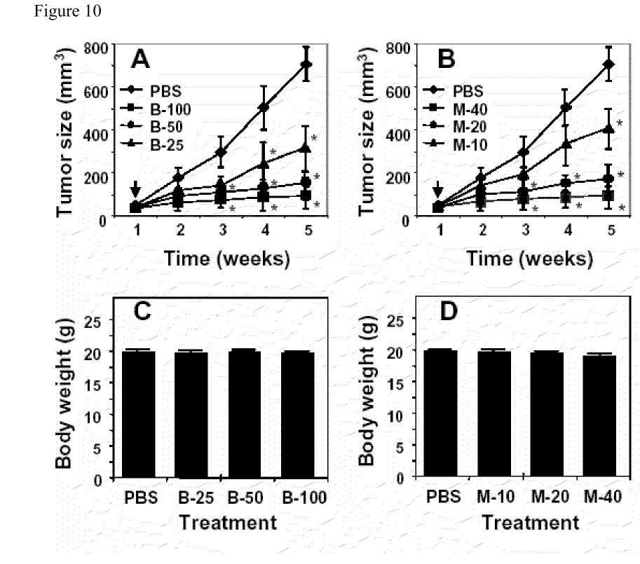

Synergistic antitumor effects of BVZ and MIT in human pancreatic cancer model. Studies have demonstrated that Sp1 is essential for VEGF expression and that VEGF plays a major role in pancreatic cancer angiogenesis. However, whether combined inhibition of both Sp1 and VEGF signaling has a synergistic antiangiogenic effect is unknown. To determine whether this takes place, we treated pancreatic cancer in nude mice with BVZ, MIT, or a combination of the two (B+M). Specifically, we first performed dose-response experiments. We injected BxPC3 cells subcutaneously into nude mice. When their tumors reached 4 mm in diameter, we gave the animals differring doses of BVZ (25, 50, and 100 μg; FIG. 10A) and MIT (0.1, 0.2, and 0.4 mg/kg; FIG. 10B) via intraperitoneal injection twice a week. We also administered PBS injections to animals as controls. We found that treatment with B+M produced dose-dependent antitumor activity. BVZ (at a dose of 25 μg) and MIT (at a dose of 0.10 mg/kg) had slight antitumor effects (P=not significant). We observed no discernable effect on body weight in the mice that received BVZ alone (FIG. 10C), suggesting that at the doses given, BVZ did not have any systemic side effects. However, at the highest dose given (0.4 mg/kg), MIT slightly reduced body weight (FIG. 10D).

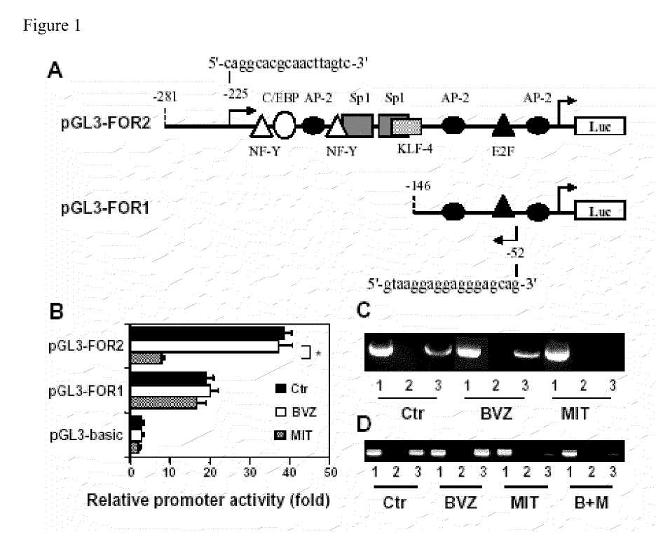

Next, we performed experiments using both BxPC3 and PANC-1 tumor cell models in which we administered PBS, BVZ (25 μg), MIT (0.10 mg/kg), or B+M to a group of nude mice. Consistently, administration of BVZ or MIT alone produced marginal antitumor activity. In contrast, administration of B+M produced synergistic antitumor activity without any systemic side effects (FIG. 1). Additionally, the mice that received B+M had body weights similar to those of the control mice that received PBS (data not shown). Therefore, the use of B+M resulted in higher antitumor activity than the use of BVZ or MIT alone did without an increase in toxicity, suggesting that treatment with B+M has a significant therapeutic benefit.

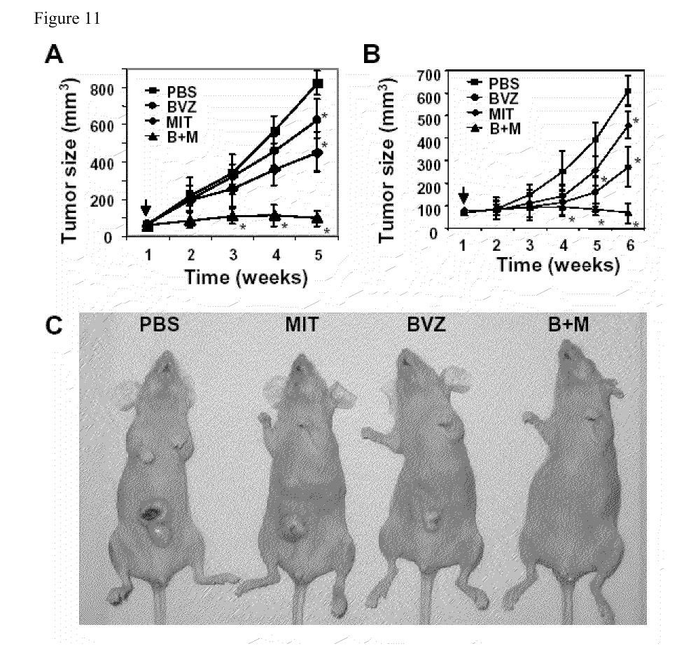

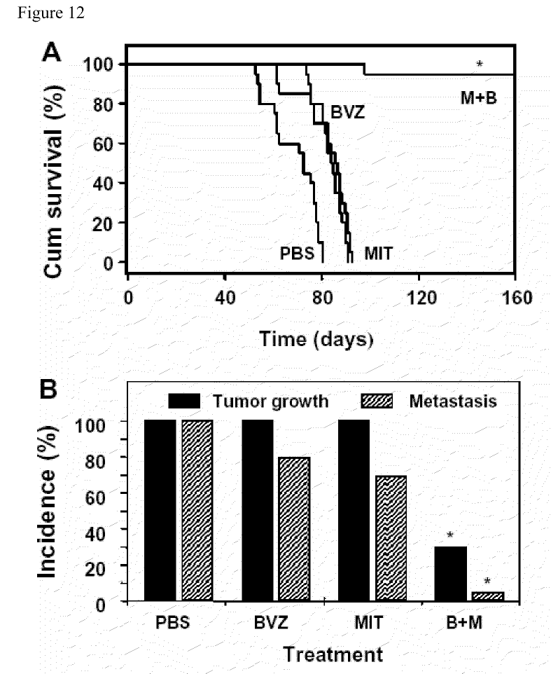

Prolonged survival of xenograft models of human pancreatic cancer that received treatment with BVZ and MIT. We injected PANC-1 cells into the pancreas of nude mice and then administered treatment to them as described in FIG. 11. We monitored animal survival daily until termination of the experiment 160 days after tumor-cell injection. We found that the mice that received BVZ or MIT alone had a slightly increased survival rate when compared with those that received PBS only, whereas the mice that received B+M had significantly longer survival (FIG. 12A). Furthermore, the incidence of tumor growth in the pancreas and of metastasis in the liver and/or other organs was significantly lower in the mice that received B+M (FIG. 12B).

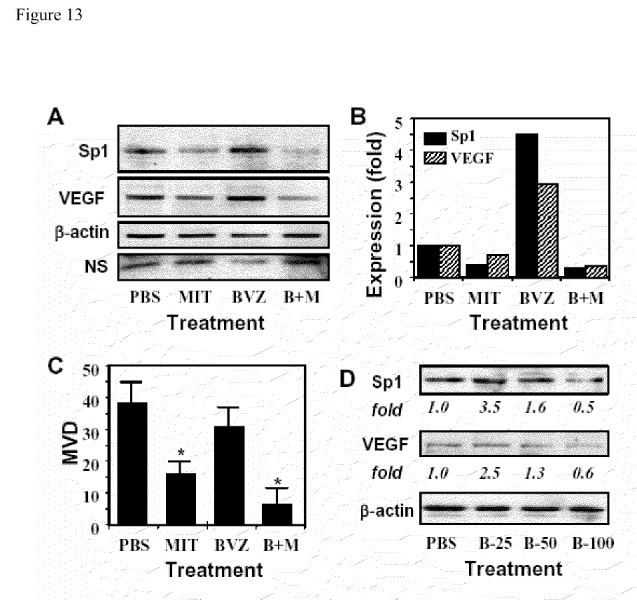

Upregulation of Sp1 and VEGF expression by treatment with BVZ and its reversal by treatment with MIT. To determine the molecular basis for the synergistic effect of treatment with BVZ and MIT, we performed Western blot analysis using total protein lysates extracted from the PANC-1 tumor tissue specimens collected from mice that received treatment with PBS, BVZ, MIT, or B+M. As shown in FIGS. 13A and 13B, treatment with BVZ alone increased expression of Sp1 and its downstream molecule VEGF. However, treatment with MIT alone suppressed Sp1 and VEGF expression, which was consistent with reduced MVD, whereas treatment with BVZ at low dose alone did not significantly reduce MVD, which was consistent with increased Sp1 expression (FIG. 13C and FIG. 13D). These data suggested that neutralization of VEGF function by BVZ may upregulate the expression of Sp1 via a positive feedback loop and lead to increased VEGF expression. However, because of this neutralization of VEGF function, the sustained MVD levels may be caused by other mechanisms. Indeed, our Western blot analysis showed that treatment with BVZ also upregulated the expression of other proangiogenic molecules, such as platelet-derived growth factor, IGF-R, and EGFR (data not shown). Therefore, BVZ resistance may result from not only overexpression of VEGF but also overexpression of other Sp1 downstream molecules.

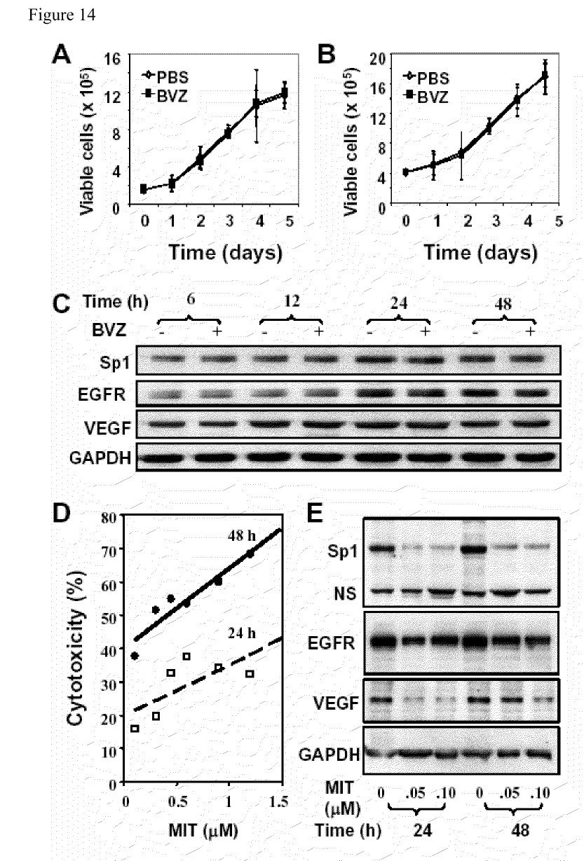

Effects of treatment with BVZ and MIT on the growth of and gene expression in human pancreatic cancer cells. To further determine whether treatment with BVZ directly impacts gene expression in pancreatic cancer cells, we incubated PANC-1 and BxPC3 cells in a medium alone or a medium containing 100 μg/ml BVZ for 6 to 48 hours. As expected, BVZ did not affect the growth of PANC-1 cells (FIG. 14A) or BxPC3 cells (FIG. 14B) in vitro. As shown in FIG. 14C, neutralization of VEGF did not affect the expression of Sp1 or its major downstream molecules, VEGF and EGFR. This result was consistent with previous findings showing that BVZ primarily blocks the autocrine effect of VEGF on tumor angiogenesis (45-49). In contrast, treatment with MIT produced dose-dependent cytotoxic effects in both PANC-1 cells (FIG. 14D) and BxPC3 cells (data not shown). The calculated IC50s for MIT were greater than 1.5 μM (24-hour assay) and 0.15 μM (48-hour assay) (FIG. 14D). Therefore, for 24 and 48 hours, we treated PANC-1 cells with MIT at concentrations of 0.05 and 0.10 μM, respectively, which were much lower than the IC50 and effectively inhibited the expression of Sp1 and its downstream molecules EGFR and VEGF (FIG. 14E). These data suggested that BVZ does not have a direct effect on tumor cells or on the expression of Sp1 and its downstream molecules but that MIT does.

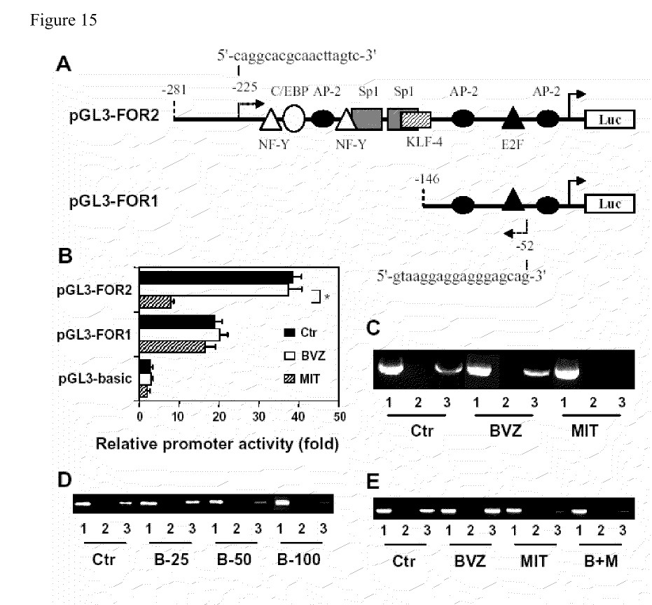

Effects of treatment with BVZ and MIT on Sp1 recruitment into the Sp1 promoter in vitro and in vivo in human pancreatic cancer cells. In this final set of experiments, we sought to determine whether treatment with BVZ and MIT regulated Sp1 expression at the transcriptional level. We transfected Sp1 promoter reporter constructs into PANC-1 cells and then incubated them in a medium alone or a medium containing 100 μg/ml BVZ or 0.1 μM MIT. In vitro, treatment with BVZ did not suppress Sp1 promoter activity, whereas treatment with MIT did. However, further deletion of Sp1-binding sites eliminated the ability of MIT to suppress Sp1 promoter activity (FIGS. 15A and 15B). Consistently, treatment with MIT significantly reduced the recruitment of Sp1 protein to its own promoter as shown in a ChIP assay (FIG. 15C). Finally, we performed a ChIP assay using tumors formed by PANC-1 cells in nude mice that received treatment as described in FIG. 11A. Although treatment with BVZ did not affect Sp1 recruitment to his own promoter in vitro (FIG. 15), it did significantly increase Sp1 recruitment to its own promoter. In contrast, both treatment with MIT alone and treatment with B+M suppressed Sp1 protein recruitment to its own promoter (FIG. 15D). These data suggested that BVZ upregulates Sp1 expression in vivo via positive feedback activation of the Sp1 gene (positive autoregulation) and then of its downstream target molecules, such as VEGF, whereas MIT disrupts this regulatory loop.

Discussion

In this study, we found that treatment with BVZ upregulated the expression of Sp1, which is a key positive regulator of the expression of various proangiogenic factors, including VEGF. Inhibition of Sp1 using MIT repressed the expression of VEGF and tumor angiogenesis. Interestingly, treatment with a combination with B+M at low doses had a synergistic antiangiogenic effect. This effect was consistent with suppression of Sp1 activity and downregulation of Sp1's multiple downstream target molecules. In summary, our experimental findings indicate that inhibition of VEGF signaling in vivo leads feedback upregulation of Sp1 expression which in turns leads to increased expression of multiple proangiogenic factors, including VEGF. Thus, treatment with anti-VEGF monoclonal antibody, BVZ, may lead to compensatory upregulation of pathways that lead to resistance. Conversely, inhibition of Sp1 blocked this feedback mechanism. Our findings not only provide researchers with a novel paradigm of synergism between antiangiogenic agents and inhibitors of transcription factors such as Sp1, but also will help clinicians design rationale combination drug therapy for improved anti-tumor activity.

Angiogenesis plays critical roles in sustained growth and metastasis of most solid tumors, including pancreatic cancer. Previous studies demonstrated that antiangiogenic therapies suppress tumor growth in animal models of pancreatic cancer. For example, several strategies inhibit the angiogenesis, growth, and metastasis of human pancreatic cancer and improve survival in nude mouse models via targeting of VEGF signaling and function, including the use of VEGF antisense oligonucleotides, VEGF-directed ribozymes, VEGF fused to a diphtheria toxin, anti-VEGF antibodies, various types of interference with VEGF receptor-1 and VEGF receptor-2, a dominant-negative flk-1, and the VEGF receptor tyrosine kinase inhibitor PTK-787 (43, 44). Additionally, celecoxib (a cyclooxygenase-2 inhibitor) and genistein (a tyrosine kinase inhibitor) suppress pancreatic cancer growth and metastasis at least in part by inhibiting VEGF expression and angiogenesis (24). Furthermore, the level of VEGF expression correlates with MVD and disease progression (4, 6). These findings from various experimental and clinical studies using various approaches substantiate the importance of the angiogenic process in pancreatic cancer and support the hypothesis that VEGF plays a crucial role in this process. Moreover, TNP-470 and endostatin reduce the rate of pancreatic cancer angiogenesis, growth, and metastasis (45).

While a number VEGF inhibitors have demonstrated significant anti-tumor activity in a wide variety xenograft models, in human studies, clinical activity for VEGF inhibitor when used as single agent has limited. Outside of renal cell carcinoma, neuroendocrine carcinoma, and some sarcomas, VEGF inhibitors are generally developed in combination. Antiangiogenic approaches have been found to be more effective when combined with chemotherapy and radiation therapy. For example, gemcitabine potentiate antiangiogenic therapies using anti-VEGF, anti-VEGF receptor-2, and anti-EGFR antibodies (46). The limited effectiveness of antiangiogenic therapies that target single molecules also highlights the important issue of diverse angiogenic signals and effector factors (6). Targeting these individual molecules individually may be ineffective and/or result in eventual resistance. In pancreatic cancer, early clinical trials of BVZ in combination with gemcitabine in advanced disease, and in combination with radiotherapy in local-regional disease have reported promising results. Various mechanisms for synergy between antiangiogenic therapy and chemotherapy have been proposed. Some have hypothesized that therapy targeting VEGF may normalize tumor vasculature, decrease intersitital fluid pressure, and enhance chemotherapy delivery. Alternatively, others have proposed that neuropilin receptors may mediate resistance to apoptosis which can be reversed by agents such as BVZ (47).

Some have recognized that elevated angiogenesis in human pancreatic cancer involves a perturbed local imbalance of proangiogenic and antiangiogenic factors, i.e., proangiogenic factors predominate over antiangiogenic factors (48, 49). Among a growing list of proangiogenic factors, various isoforms of VEGF contribute to the growth and metastasis of pancreatic cancer through a variety of mechanisms (4, 6). Moreover, pancreatic cancer cells overexpress several other mitogenic growth factors that are also angiogenic, such as EGF, transforming growth factor-α, HGF, FGFs (e.g., FGF-1, FGF-2, FGF-5), and platelet-derived growth factor-β (4, 6). Researchers have shown that many of these and other factors correlate with increased vessel formation and poor prognosis in patients with pancreatic cancer; this includes expression of FGF-2 and platelet-derived endothelial cell growth factor, mutation of K-ras, and overexpression of hypoxia-inducible factor-1a, thymidine phosphorylase, thrombospondin-1, and cathepsin B and L (4-6). Human pancreatic cancer cells also overexpress interleukin-8, and a previous study showed that specific neutralizing antibodies against interleukin-8 significantly reduce the growth and metastasis of human pancreatic cancer in orthotopic animal models (50). Therefore, whereas VEGF is crucial for promoting the growth and metastasis of pancreatic cancer, other factors are likely involved in this process, as well. Together, these factors may produce mitogenic activity in an autocrine and paracrine fashion, promoting pancreatic tumor-cell growth and angiogenesis and eventually enhancing pancreatic tumor invasion and metastasis (4-6). This notion is supported by the fact that interference with the expression and function of these factors influences the angiogenesis and growth of human pancreatic cancer in animal models. For example, antiangiogenesis can be achieved using treatment with dominant-negative IGF-IR; antithrombin III and vitamin D-binding protein-macrophage activating factor; NK4, a four-kringle fragment of HGF; and the tyrosine kinase inhibitor of EGFR (4, 6). However, simultaneously interfering with multiple target molecules is clinically challenging.

Recent studies have suggested that there is a potential underlying mechanism for overexpression of various proangiogenic factors that collectively regulate pancreatic cancer angiogenesis (6, 9). For example, in previous studies, we showed that Sp1 is constitutively activated in human pancreatic cancer cells and that constitutive Sp1 activity is essential for constitutive, inducible VEGF expression (6, 9, 23, 27, 28). In our previous studies, we also found that overexpression of Sp1 is correlated with MVD in human gastric cancer tissue and that manipulating the Sp1 expression level using a small interfering RNA approach significantly inhibits the tumor angiogenic phenotype (6, 9). This antiangiogenic effect of knocking down Sp1 expression is consistent with reduced expression of several signaling molecules in the signaling pathways that play important roles in the regulation of tumor angiogenesis (28-30). In fact, one of our recent studies showed that inhibition of VEGF expression and the resulting antitumor effect of celecoxib are mediated at least in part by suppression of Sp1 activity (24). All of these lines of evidence indicate that Sp1 plays an important role in regulation of angiogenesis in human pancreatic cancer. Furthermore, as shown in the present study, blockade of Sp1 activity by MIT suppresses tumor angiogenesis.

Finally, treatment with a low dose of MIT did not significantly inhibit tumor angiogenesis in the present study, although the overall level of VEGF protein expression in tumors was reduced. This finding suggests that the quantity of extracellular matrix-associated VEGF in tumor bed may be sufficient to initiate and maintain the tumor angiogenesis phenotype without continuing production of VEGF in tumor bed. Removal of this angiogenic signal produced by the existing VEGF is necessary, which may explain why a single treatment with a low dose of MIT is insufficient to eliminate angiogenesis in pancreatic tumors. On the other hand, the functional removal of VEGF by treatment with BVZ upregulates the expression of many angiogenic molecules other than VEGF, which may explain why a single treatment with BVZ was insufficient to produce sustained antiangiogenesis. The combined use of both BVZ and MIT should produce sustained antiangiogenesis in pancreatic cancer. This is clearly supported by the present study, in which combined use of low doses of BVZ and MIT had a synergistic antiangiogenic effect in human pancreatic cancer models. Therefore, our present findings underscore the important roles of Sp1 and VEGF in pancreatic cancer angiogenesis.

Collectively, our study suggests that BVZ treatment targeting VEGF may lead to resistance of pancreatic cancer to BVZ in part via positive feedback activation of the transcription factor Sp1 and subsequent overexpression of Sp1's downstream target genes. The use of mithramycin A in combination with BVZ represents an important novel strategy of targeting angiogenesis by sequentially interfering with upstream transcriptional regulation by Sp1 and neutralizing of downstream effector molecule VEGF. Combining bevacizumab and mithramycin A in human clinical studies presents a rationale way forward for the development of antiangiogenic therapy.

### REFERENCES

1 Jemal A, Murray T, Ward E, et al. Cancer statistics, 2005. CA Cancer J Clin 2005; 55:10-30.
2. Kuvshinoff B W, Bryer MP. Treatment of resectable and locally advanced pancreatic cancer. Cancer Control 2000; 7:428-36.
3. MacKenzie M J. Molecular therapy in pancreatic adenocarcinoma. Lancet Oncol 2004; 5:541-9.
4. Korc M. Pathways for aberrant angiogenesis in pancreatic cancer. Mol Cancer 2003; 2:8.
Kern SE. Molecular genetic alterations in ductal pancreatic adenocarcinomas. Med Clin North Am 2000; 84:691-5.
6. Xie K, Wei D, Huang S. Transcriptional anti-angiogenesis therapy of human pancreatic cancer. Cytokine Growth Factor Rev 2006; 17:147-56.
7. Folkman J. Angiogenesis. Annu Rev Med 2006; 57:1-18.
8. Kerbel R S. Antiangiogenic therapy: a universal chemosensitization strategy for cancer? Science. 2006; 312:1171-5.
9. Xie K, Wei D, Shi Q, Huang S. Constitutive and inducible expression and regulation of vascular endothelial growth factor. Cytokine Growth Factor Rev 2004; 15:297-324.
10. Presta L G, Chen H, O'Connor S J, et al. Humanization of an anti-vascular endothelial growth factor monoclonal antibody for the therapy of solid tumors and other disorders. Cancer Res 1997; 57:4593-9.
11. Dvorak H F. Discovery of vascular permeability factor (VPF). Exp Cell Res 2006; 312(5):522-6.
12. Ellis L M, Takahashi Y, Fenoglio C J, Cleary K R, Bucana C D, Evans D B. Vessel counts and vascular endothelial growth factor expression in pancreatic adenocarcinoma. Eur J Cancer 1998; 34:337-40.
13. Fujimoto K, Hosotani R, Wada M, et al. Expression of two angiogenic factors, vascular endothelial growth factor and platelet-derived endothelial cell growth factor in human pancreatic cancer, and its relationship to angiogenesis. Eur J Cancer 1998; 34:1439-47.
14. Ikeda N, Adachi M, Taki T, et al. Prognostic significance of angiogenesis in human pancreatic cancer. Br J Cancer 1999; 79:1553-63.
15. Karademir S, Sokmen S, Terzi C, et al. Tumor angiogenesis as a prognostic predictor in pancreatic cancer. J Hepatobiliary Pancreat Surg 2000; 7:489-95.
16. Couvelard A, O'Toole D, Leek R, et al. Expression of hypoxia-inducible factors is correlated with the presence of a fibrotic focus and angiogenesis in pancreatic ductal adenocarcinomas. Histopathology 2005; 46:668-76.
17. Hotz H G, Hines O J, Masood R, et al. VEGF antisense therapy inhibits tumor growth and improves survival in experimental pancreatic cancer. Surgery 2005; 137:192-9.
18. McBride G. Researchers optimistic about targeted drugs for pancreatic cancer. J Natl Cancer Inst 2004; 96:1570-2.
19. Hakam A, Fang Q, Karl R, Coppola D. Coexpression of IGF-1R and c-Src proteins in human pancreatic ductal adenocarcinoma. Dig Dis Sci 2003; 48:1972-8.
20. Tomioka D, Maehara N, Kuba K, et al. Inhibition of growth, invasion, and metastasis of human pancreatic carcinoma cells by NK4 in an orthotopic mouse model. Cancer Res 2001; 61:7518-24.
21. Maehara N, Matsumoto K, Kuba K, Mizumoto K, Tanaka M, Nakamura T. NK4, a four-kringle antagonist of HGF, inhibits spreading and invasion of human pancreatic cancer cells. Br J Cancer 2001; 84:864-73.
22. Kornmann M, Beger H G, Korc M. Role of fibroblast growth factors and their receptors in pancreatic cancer and chronic pancreatitis. Pancreas 1998; 17:169-75.
23. Shi Q, Le X, Abbruzzese J L, et al. Constitutive Sp1 activity is essential for differential constitutive expression of vascular endothelial growth factor in human pancreatic adenocarcinoma. Cancer Res 2001; 61:4143-54.
24. Wei D, Wang L, He Y, et al. Celecoxib inhibits vascular endothelial growth factor expression in and reduces angiogenesis and metastasis of human pancreatic cancer via suppression of Sp1 transcription factor activity. Cancer Res 2004; 64:2030-8.
25. Black A R, Black J D, Azizkhan-Clifford J. Sp1 and kruppel-like factor family of transcription factors in cell growth regulation and cancer. J Cell Physiol 2001; 188:143-60.
26. Li L, He S, Sun J M, Davie J R. Gene regulation by Sp1 and Sp3. Biochem Cell Biol 2004; 82:460-71.
27. Mukhopadhyay D, Datta K. Multiple regulatory pathways of vascular permeability factor/vascular endothelial growth factor (VPF/VEGF) expression in tumors. Semin Cancer Biol 2004; 14:123-30.
28. Abdelrahim M, Smith R, 3rd, Burghardt R, Safe S. Role of Sp proteins in regulation of vascular endothelial growth factor expression and proliferation of pancreatic cancer cells. Cancer Res 2004; 64:6740-9.
29. Safe S, Abdelrahim M. Sp transcription factor family and its role in cancer. Eur J Cancer 2005; 41:2438-48.
30. Wang L, Wei D, Huang S, et al. Transcription factor Sp1 expression is a significant predictor of survival in human gastric cancer. Clin Cancer Res 2003; 9:6371-80.
31. Wohlert S E, Kunzel E, Machinek R, Mendez C, Salas J A, Rohr J. The structure of mithramycin reinvestigated. J Nat Prod 1999; 62:119-21.
32. Prado L, Lombo F, Brana A F, Mendez C, Rohr J, Salas J A. Analysis of two chromosomal regions adjacent to genes for a type II polyketide synthase involved in the biosynthesis of the antitumor polyketide mithramycin in Streptomyces argillaceus. Mol Gen Genet. 1999; 261:216-25.
33. Majee S, Dasgupta S D, Chakrabarti A. Interaction of the DNA-binding antitumor antibiotics, chromomycin and mithramycin with erythroid spectrin. Eur J Biochem 1999; 260:619-626.
34. Hall T J, Schaeublin M, Chambers T J. The majority of osteoclasts require mRNA and protein synthesis for bone resorption in vitro. Biochem Biophys Res Commun 1993; 195:1245-53.
35. Remsing L L, Bahadori H R, Carbone G M, McGuffie E M, Catapano C V, Rohr J. Inhibition of c-src transcription by mithramycin: structure-activity relationships of biosynthetically produced mithramycin analogues using the c-src promoter as target. Biochemistry 2003; 42:8313-24.
36. Chatterjee S, Zaman K, Ryu H, Conforto A, Ratan R R. Sequence-selective DNA binding drugs mithramycin A and chromomycin A3 are potent inhibitors of neuronal apoptosis induced by oxidative stress and DNA damage in cortical neurons. Ann Neurol 2001; 49:345-354.
37. Blume S W, Snyder R C, Ray R, Thomas S, Koller C A, Miller D M. Mithramycin inhibits SP1 binding and selectively inhibits transcriptional activity of the dihydrofolate reductase gene in vitro and in vivo. J Clin Invest 1991; 88:1613-21.
38. Tagashira M, Kitagawa T, Isonishi S, Okamoto A, Ochiai K, Ohtake Y. Mithramycin represses MDR1 gene expression in vitro, modulating multidrug resistance. Biol Pharm Bull 2000; 23:926-9.
39. Duverger V, Murphy A M, Sheehan D, et al. The anticancer drug mithramycin A sensitises tumour cells to apoptosis induced by tumour necrosis factor (TNF). Br J Cancer 2004; 90:2025-31.
40. Koutsodontis G, Kardassis D. Inhibition of p53-mediated transcriptional responses by mithramycin A. Oncogene 2004; 23:9190-200.
41. Weidner N, Semple J P, Welch W R, Folkman J. Tumor angiogenesis and metastasis—correlation in invasive breast carcinoma. N Engl J Med 1991; 324:1-8.
42. Kanai M, Wei D, Li Q, et al. Kruppel-like Factor 4 Represses Sp1 Expression and Contributes to Its Tumor Suppressor Activity in Human Pancreatic and Gastric Cancer. Clin Cancer Res 2006; in press.
43. Morabito A, De Maio E, Di Maio M, Normanno N, Perrone F. Tyrosine kinase inhibitors of vascular endothelial growth factor receptors in clinical trials: current status and future directions. Oncologist 2006; 11:753-64.
44. Yazici Y D, Kim S, Jasser S A, et al. Antivascular therapy of oral tongue squamous cell carcinoma with PTK787. Laryngoscope 2005; 115:2249-55.
45. Prox D, Becker C, Pirie-Shepherd S R, Celik I, Folkman J, Kisker O. Treatment of human pancreatic cancer in mice with angiogenic inhibitors. World J Surg 2003; 27:405-11.
46. Wiedmann M W, Caca K. Molecularly targeted therapy for gastrointestinal cancer. Curr Cancer Drug Targets 2005; 5:171-93.
47. Kowanetz M, Ferrara N. Vascular endothelial growth factor signaling pathways: therapeutic perspective. Clin Cancer Res 2006; 12:5018-22.
48. Jain R K. Tumor angiogenesis and accessibility: role of vascular endothelial growth factor. Semin Oncol 2002; 29:3-9.
49. Blagosklonny M V. How Avastin potentiates chemotherapeutic drugs: action and reaction in antiangiogenic therapy. Cancer Biol Ther 2005; 4:1307-10.
50. Xie K. Interleukin-8 and human cancer biology. Cytokine Growth Factor Rev 2001; 12:375-91.

Conclusion

The activity of mithramycin was studied in a variety of human cancer xenograft models including carcinoid tumors, pancreatic cancer, and ovarian cancer. In as series of studies, we evaluated the dose of mithramycin required for Sp1 inhibition as well as the duration of Sp1 inhibition following administration of mithramycin. We found that dose of mithramycin required for inhibition of Sp1 in nude-mice human cancer xenograft models to be significantly lower then the dose use for other indications in mice experiments. The duration of Sp1 inhibition is 24 hours (FIG. 6).

Our data suggest that the previously used MTD based method for dosing mithramycin in the clinics is not optimal for anti-angiogenic activity. Mithramycin at significantly lower doses given weekly or twice weekly can inhibit Sp1 in a continuous manner with lower toxicity. Further, in our experiments mithramycin had significant activity when given either intraperitoneally or subcutaneously (FIG. 5) suggesting that alternative dosing methods could be used in future clinical studies.

Further, synergistic anti-tumor activity was observed when mithramycin was combined with other agents targeting angiogenesis such as VEGF inhibitor bevcaizumab. Details of experiments in pancreatic cancer, ovarian cancer, and carcinoid tumors are attached or outlined below.

### Example 6

### Inhibition of SP1 Expression in Growing Tumors by Mithramycin A Directly Correlates with its Potent Antiangiogenic Effects in Human Neuroendocrine Xenograft Models

Abbreviations used: EGFP, enhanced green fluorescent protein; EGFR, epidermal growth factor receptor; MOI, multiplicity of infection; CI, confidence interval; PBS, phosphate-buffered saline; PDGFA, platelet-derived growth factor alpha; MVD, microvessel density; VEGF, vascular endothelial growth factor; ELISA, enzyme-linked immunoassay.

Abstract

Our previous studies have shown that human neuroendocrine tumor cells overexpress pro-angiogenic factors, vascular endothelial growth factor A (VEGF), and transcription factor Sp1 expression correlates with VEGF expression. However, the impact of anti-angiogenic therapy on the Sp 1VEGF pathway remains unknown. Treatment with bevacizumab (BVZ), a neutralizing antibody against VEGF, suppressed human neuroendocrine tumor growth in nude mice in a dose-dependent manner. Gene expression analyses revealed that ineffective doses substantially upregulated the expression of Sp1 and its downstream target genes, including VEGF, EGFR and PDGFA, in tumor tissues. Treatment with mithramycin A, a Sp1 inhibitor, suppressed the expression of Sp1 and its downstream target genes in both cell culture and tumors growing in nude mice. Combined treatment with BVZ and mithramycin A produced synergistic tumor suppression, which was consistent with suppression of the expression of Sp1 and its downstream target genes. Thus, treatment with BVZ may block VEGF function but activate the pathway of its expression via positive feedback. Given the notion that Sp1 is an important regulator of the expression of multiple angiogenic factors, BVZ-initiated upregulation of Sp1 and subsequent overexpression of Sp1's downstream target genes may profoundly affect the effectiveness of anti-angiogenic strategies for human neuroendocrine tumors. Combination therapy with BVZ and MIT blocks this resistance mechanism and represents a promising novel approach to enhancing the activity of anti-angiogenic therapy.

Introduction

Carcinoid is derived from the term “Karzinoide” which Obemdorfer first used in 1907 to describe low to intermediate grade neuroendocrine tumors (LGNETs) that had a more indolent clinical course than the more common adenocarcinomas (1). These tumors originate from the neuroendocrine cells throughout the body and are capable of producing a variety of hormones and biogenic amines. Once thought to be rare, recent analyses of the SEER database have shown a steady increase in reported incidence of LGNETs (2, 3). Our recent analysis suggests that because the longer survival experienced by many patients, the prevalence of LGNETs in the United States exceeds 100,000 cases (3).

LGNETs have a wide range of aggressiveness. For loco-regional disease, a unified staging system is missing. Disease is incurable in the metastatic setting. While some patients live for months following the diagnosis of metastatic disease, others live beyond 8 years. Somatostatin analogues are commonly used to control hormonal related symptoms, but tumor regression is rare. They are generally resistant to cytotoxic chemotherapy (4). Systemic therapy options are lacking.

Inherited mutations in several tumor suppressor genes including MEN1, vHL, TS2, and NF1 have been associated with development of LGNETs (5-9). The genetic abnormalities associated with carcinogenesis and progression in sporadic LGNETs are less well understood; however, allelic deletions at chromosome 11q13 and 3p25 (the sites of the MEN1 and vHL genes) have been observed (10-13). LGNETs are highly vascular tumors often with accompanying desmoplastic reaction. Previous studies indicated the role of several growth factor families in carcinod development and progression, including bFGF, TGF, EGFR, IGFR, and VEGF (14-19). However, the molecular mechanisms behind the abnormal expression of multiple molecules remain unclear. Our previous study of human gastric and pancreatic cancer and LGNETs suggests that abnormal Sp1 activation augments the angiogenic and metastatic capacity of tumor cells through overexpression of multiple Sp1 downstream genes, including the key angiogenic factor VEGF (20, 21). Our recent studies demonstrated that the transcription factor Sp1 plays an important role in regulating expression of VEGF in human LGNET (22). However, it is unclear whether the expression of Sp1 predicts the angiogenic phenotype of human LGNET and whether Sp1 is a reliable anti-angiogenic target.

Sp1 is a zinc finger transcription factor that is important to the transcription of many cellular and viral genes that contain GC boxes in their promoters. Researchers have cloned additional transcription factors similar to Sp1 in their structural and transcriptional properties (Sp2, Sp3, and Sp4), thus forming the Sp1 multigene family (23). Although Sp1 has been perceived to be a basal transcription factor since its discovery, increasing evidence suggests that it regulates a variety of biological functions, including cell survival, growth, differentiation, tumor-genesis, and progression (23-27). If Sp1 overexpression is directly correlated with the angiogenic potential (28), Sp1 inhibitors such as mithramycin-A (MIT) may have profound anti-angiogenic effects for human LGNETs.

MIT, also known as aureolic acid and plicamycin (Mithracin), is an aureolic acid-type polyketide produced by various soil bacteria of the genus Streptomyces (29, 30). In the past, MIT has been used to manage malignant hypercalcemia, Paget's disease, and various malignancies including testicular carcinoma, chronic myeloid leukemia, and acute myeloid leukemia (31-33). MIT binds to GC-rich regions in chromatin and interferes with the transcription of genes that bear GC-rich motifs in their promoters (31, 34). Its mechanism of action involves a reversible interaction with double-stranded DNA with GC-based specificity. MIT is believed to act, in part, by selectively regulating transcription of genes that have GC-rich promoter sequences (35). In addition, recent studies have shown that MIT sensitizes tumor cells to apoptosis induced by tumor necrosis factor and inhibits p53-mediated transcriptional responses (36, 37). Inhibition of Sp1 activity is considered to be a major mechanism of the antitumor activity of MIT. Thus, we performed the present study to determine the effect of MIT as anti-angiogenic therapy for human LGNETs.

Materials and Methods

Chemicals and Reagents. Mithramycin-A (MIT, 1 mg/vial crystal powder; lot 055K4011) was purchased from Sigma Chemical Co. (St. Louis, Mo.) and diluted in sterile water. BVZ (25 mg/ml; NDC 50242-060-01) from Genentech, Inc. (South San Francisco, Calif.) was purchased. For animal experiments, MIT (0.1 to 0.4 mg/kg body weight) and BVZ (25 to 100 μg/mouse) were administered by intraperitoneal or subcutaneous injections twice a week or as otherwise stated.

Cell Lines and Culture Conditions. The human bronchial LGNET cancer cell line NCI-H727 (ATCC: CRL-5815) was purchased from the American Type Culture Collection (ATCC, Manassas, Va.). Human pancreatic LGNET cell line BON-[(38) was provided by Dr. Courtney M. Townsend (Galveston, Tex.) to Dr. Funda Meric-Bernstam. All of the cell lines were maintained in plastic flasks as adherent monolayers in MEM supplemented with 10% fetal bovine serum, sodium pyruvate, nonessential amino acids, L-glutamine, and a vitamin solution (Flow Laboratories, Rockville, Md.).

Animals. Female athymic BALB/c nude mice were purchased from The Jackson Laboratory (Bar Harbor, Me.). The mice were housed in laminar flow cabinets under specific pathogen-free conditions and used when they were 8 weeks old. The animals were maintained in facilities approved by the Association for Assessment and Accreditation of Laboratory Animal Care in accordance with the current regulations and standards of the United States Department of Agriculture, Department of Health and Human Services, and NIH.

Western blot analysis. Whole-cell lysates were prepared from human LGNET cell lines and tissues (21). Standard Western blotting was performed using polyclonal rabbit antibodies against human and mouse Sp1, VEGF, EGFR and PDGFA (Santa Cruz Biotechnology, Santa Cruz, Calif.) and the anti-rabbit IgG antibody, a horseradish peroxidase-linked F(ab′)₂ fragment obtained from a donkey (Amersham, Arlington Heights, Ill.). Equal protein-sample loading was monitored by probing the same membrane filter with an antibody against glyceraldehyde-3-phosphate dehydrogenase (GAPDH) (21). The probe proteins were detected using the Amersham enhanced chemiluminescence system according to the manufacturer's instructions.

Chromatin immunoprecipitation. Chromatin was prepared from cells and tumors as described previously (39). Chromatin immunoprecipitation (ChIP) assay was performed using the Chromatin Immunoprecipitation Assay Kit (Upstate Cell Signaling Solutions, Lake Placid, N.Y.) according to the manufacturer's instructions. Briefly, DNA cross-binding proteins were cross-linked with DNA and lysed in sodium dodecyl sulfate lysis buffer. The lysate was sonicated to shear DNA to 200 to 500 bp. After preclearing with a salmon sperm DNA/protein A agarose-50% slurry for 30 minutes at 4° C., chromatin samples were immunoprecipitated overnight with no antibody or an anti-Sp1 antibody (PEP2). The region between −224 and -53 bp of the Sp1 promoter was amplified using the following primers: sense, 5′-caggcacgcaacttagtc-3′, and antisense, 5′-gtaaggaggagggagcag-3′. Polymerase chain reaction products were separated on a 1.5% agarose gel, stained with ethidium bromide, and visualized under ultraviolet light.

Immunohistochemical analysis and quantification of tumor MVD. For CD31 staining, frozen tissue sections (5 μm thick) were fixed in acetone. Endogenous peroxidase in the specimens was blocked using 3% hydrogen peroxide in phosphate-buffered saline (PBS) for 12 minutes. The specimens were incubated for 20 minutes at room temperature in a protein-blocking solution consisting of PBS (pH 7.5) containing 5% normal horse serum and 1% normal goat serum and then incubated overnight at 4° C. in a 1:100 dilution of monoclonal goat anti-CD31 (PECAM1-M20), polyclonal rabbit anti-Sp 1, or polyclonal rabbit anti-VEGF, -EGFR and —PDGFA antibodies (Santa Cruz Biotechnology). The specimens were then rinsed and incubated with peroxidase-conjugated anti-goat or anti-rabbit IgG for 1 hour at room temperature. Next, the slides were rinsed with PBS and incubated with diaminobenzidine (Research Genetics, Huntsville, Ala.) for 5 minutes. Frozen sections of the specimens were then washed three times with distilled water, counterstained with Mayer's hematoxylin (Biogenex Laboratories, San Ramon, Calif.), and washed once each with distilled water and PBS. The slides were mounted with Universal Mount (Research Genetics) and examined under a bright-field microscope. A positive reaction was indicated by a reddish-brown precipitate in the cytoplasm (CD31) or nuclei (Sp1). For quantification of tumor MVD, vessels on each section were counted in five high-power fields (magnification, ×200 [×20 objective and ×10 ocular]) as described previously (40).

Statistical analysis. Each experiment was performed independently at least twice with similar results; one representative experiment is presented. The significance of the in vitro data was determined using Student's t-test (two-tailed), whereas the significance of the in vivo data was determined using the two-tailed Mann-Whitney Utest. P≦0.05 was deemed significant.

Results

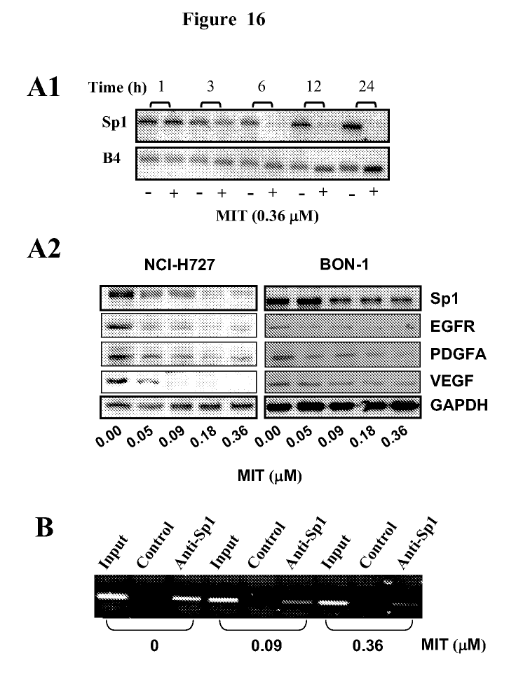

MIT inhibited Sp1 expression and the expression of its downstream target molecules in human LGNET cells. Our earlier studies have shown that Sp1 plays an important role in LGNET development and progression, particularly angiogenesis, given the notion that Sp1 regulates the expression of multiple factors key to tumor angiogenesis. In the first set of experiments, we sought to determine the effect of MIT on Sp1 and its downstream target molecules in human LGNET cancer cells. H727 cells were incubated in medium or medium containing 0.36 μM MIT for 24 hours. mRNA was extracted for Northern blot analysis of Sp1 expression (FIG. 16A1). H727 and BON-1 cells were incubated in medium or medium containing different concentrations of MIT for 1 to 24 hours. Protein extracts were used for Western blot analyses of expression of Sp1 and its downstream molecules. As shown in FIG. 16A2, MIT produced a concentration-dependent inhibition of Sp1 protein and other proteins. To further determine whether MIT removes Sp1 from Sp1-binding sites, we performed the ChIP assay. As shown in FIG. 16B, MIT compete with Sp1 binding to the Sp1-binding sites in the promoters of Sp1, VEGF, EGFR and PDGFA genes. Our studies have demonstrated that MIT inhibits Sp1 expression and its downstream molecules including VEGF, EGFR and PDGFA.

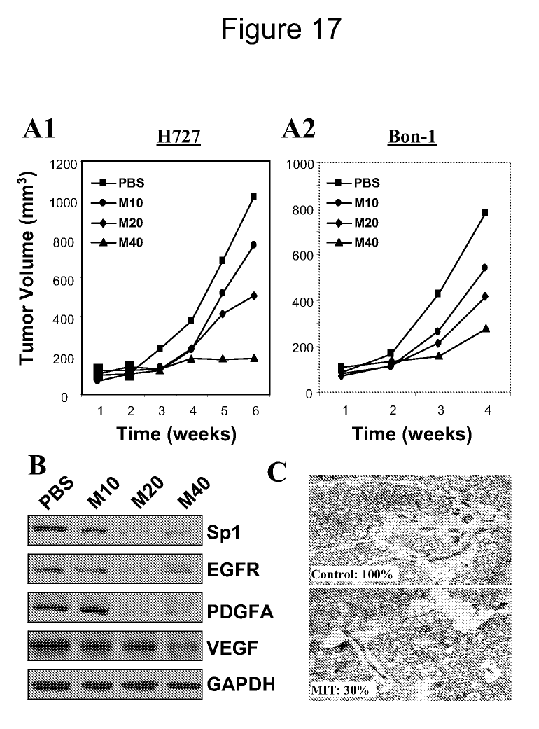

MIT inhibited angiogenesis of human LGNET in animal models. In the next set of experiments, we determined whether MIT treatment inhibits LGNET growth in mouse models. H727 and BON-1 tumor cells were injected into the subcutis of mice as described in the Materials and Methods. MIT treatment started when tumor sizes reached around 3-4 mm in diameters. As shown in FIG. 2A, MIT treatment produced dose-dependent inhibition of tumor growth in mouse models. Moreover, the residual tumors were harvested and angiogenesis was assessed by CD31 staining. As shown in FIG. 2B, MIT treatment substantially inhibited Sp1 expression and the expression of VEGF, EGFR and PDGFA. Moreover, drastic inhibitions of expression of Sp1 and its downstream targets were consistent with a reduced MVD (FIG. 17C). These data suggested that inhibition of Sp1 expression by MIT exhibited a marked anti-angiogenesis and antitumor activity.

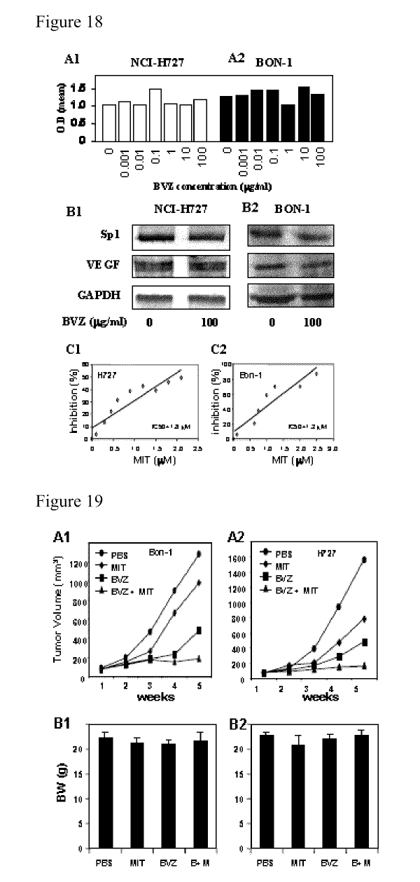

Effects of treatment with BVZ and MIT on the growth of and gene expression in human LGNET cells. To further determine whether treatment with BVZ directly impacts gene expression in LGNET cells, we incubated H727 and BON-1 cells in a medium alone or a medium containing 0 to 100 μg/ml BVZ for 5 days. As expected, BVZ did not affect the growth of H727 and Bon-1 cells (FIG. 18A) in vitro. As shown in FIG. 18B, neutralization of VEGF did not affect the expression of Sp1 or its major downstream molecule, VEGF. In contrast, treatment with MIT produced dose-dependent cytotoxic effects in both H727 and BON-1 cells (FIG. 18C). The calculated IC50s for MIT were greater than 1.8 μM (H727 cells) and 0.12 μM (Bon-1 cells) (FIG. 18C). MIT at concentrations of 0.05 to 0.36 μM, which were much lower than the IC50 and effectively inhibited the expression of Sp1 and its downstream molecules EGFR, PDGFA, and VEGF (FIG. 16A). These data suggested that BVZ does not have a direct effect on tumor cells or on the expression of Sp1 and its downstream molecules but that MIT does.

Synergistic antitumor effects of BVZ and MIT in human LGNET model. The above studies may suggest that neutralization of VEGF by BVZ and corresponding reduction of blood perfusion activates the expression of Sp1 (positive feedback) and its downstream molecule VEGF, given the essential role of Sp1 in VEGF expression. To test this hypothesis, we determined whether modulating Sp1 expression and/or activity impacts the efficacy of anti-angiogenic treatment of BVZ. Specifically, we injected H727 and BON-1 cells subcutaneously into nude mice. When their tumors reached 4 mm in diameter, we gave the animals different doses of BVZ (25 100 μg) alone or in combination with MIT (0.10 mg/kg, with slight antitumor effects) via intraperitoneal injection twice a week. We also administered PBS injections to animals as controls. We found that administration of BVZ or MIT alone produced marginal antitumor activity whereas combined use of BVZ at a dose of 25 μg and MIT at a dose of 0.10 mg/kg produced a synergistic antitumor activity (FIG. 19A). We observed no discernable effects on body weight in the mice that received BVZ alone (FIG. 19B), suggesting that at the doses given, BVZ did not have any systemic side effects. However, at the highest dose given (0.4 mg/kg), MIT slightly reduced body weight (data not shown). Additionally, the mice that received combination of BVZ and MIT had body weights similar to those of the control mice that received PBS (data not shown). Therefore, the combined use of BVZ and MIT resulted in higher antitumor activity than the use of BVZ or MIT alone did without an observed increase in toxicity, suggesting that the combination treatment has a significant therapeutic benefit.

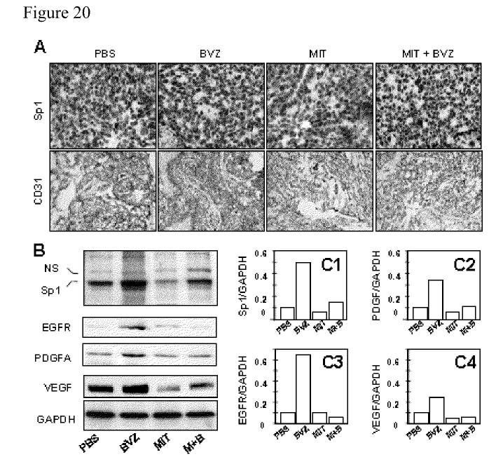

Prolonged survival of xenograft models of human LGNET that received treatment with BVZ and MIT. H727 and BON-1 cells were injected subcutaneously into nude mice and then administered treatment to them as described in FIG. 20. We monitored animal survival daily until termination of the experiment 160 days after tumor-cell injection. We found that the mice that received BVZ or MIT alone had a slightly increased survival rate when compared with those that received PBS only, whereas the mice that received BVZ and MIT had significantly longer survival. Furthermore, the incidence and extent of tumor growth was significantly lower in the mice that received BVZ and MIT (data not shown).

Upregulation of Sp1 and VEGF expression by treatment with BVZ and its reversal by treatment with MIT. To determine the molecular basis for the synergistic effect of treatment with BVZ and MIT, we performed a Western blot analysis using total protein lysates extracted from the H727 tumor tissue specimens collected from mice that received treatment with PBS, BVZ, MIT, or B+M. As shown in FIG. 20A treatment with BVZ alone increased expression of Sp1. However, treatment with MIT alone suppressed Sp1 expression, which was consistent with reduced MVD, whereas treatment with BVZ at a low dose alone did not significantly reduce MVD, which was consistent with increased Sp1 expression. Indeed, our Western blot analysis showed that treatment with BVZ also upregulated the expression of other proangiogenic molecules, such as PDGFA, VEGF, and EGFR (FIGS. 20B and 20C). These data suggest that neutralization of VEGF function by BVZ may upregulate the expression of Sp1 via a positive feedback loop and lead to increased VEGF expression and upregulation of Sp1 downstream target genes that are key to angiogenesis. Therefore, BVZ resistance may result from not only overexpression of VEGF but also from overexpression of other Sp1 downstream molecules.

Discussion

In this study, we found that treatment with BVZ up-regulated the expression of Sp1, which is a key positive regulator of several proangiogenic pathways, including VEGF, EGF, and PDGF. Inhibition of Sp1 using MIT repressed the expression of VEGF, EGFR, PDGFA, and tumor angiogenesis. Interestingly, treatment with a combination with BVZ and MIT at low doses had a synergistic anti-angiogenic effect. This effect was consistent with suppression of Sp1 activity and downregulation of Sp1's multiple downstream target molecules.

It is well recognized that angiogenesis is required for tumor growth beyond a small size and is crucial to the process of metastasis. Angiogenesis plays critical roles in sustained growth and metastasis of human LGNETs which are known to be particularly vascular. VEGF, a potent promoter of angiogenesis, is expressed in both gastrointestinal and pulmonary carcinoid tumors (19, 41). Furthermore, recent studies have demonstrated the expression of VEGF receptor (VEGFR)—FLK and VEGFR-FLT1 in carcinoid tumor cells (42). Two studies recently examined the prognostic value of VEGF in patients with neuroendocrine tumors. In the first study, the investigators found that VEGF expression and microvessel density were higher in patients with more well-differentiated neuroendocrine tumors than in those with less-differentiated tumors (43). Also, they observed progressive loss of VEGF expression in the more poorly differentiated tumors. This suggests that VEGF has a significant role in the biologic behavior of LGNETs. In the second study, we examined the effect of VEGF expression in LGNETs only (22). Our prior study has demonstrated that VEGF expression directly correlated with microvessel density, metastasis, and poor progression-free survival among patients with LGNET (22). In a recent human phase II study of BVZ in LGNETs, we demonstrated that BVZ therapy was associated with decrease in tumor blood flow and improved progression free survival. Similarly, VEGF tyrosine kinase inhibitors including sunitinib (44), and sorafinib (45) have also demonstrated activity in LGNETs. However, in these studies and others, VEGF inhibition is not curative and resistance to VEGF inhibitors eventually develops in most patients. While the mechanisms of resistance are not fully understood, data from clinical and laboratory studies suggests that upregulation of proangiogenic pathways may play a crucial role. For example, in a mouse model of LGNET, investigator found VEGF receptor-2 inhibition led to upregulation of VEGF, FGF2, and angiopoietin (46). Further, same investigators were able to reproduce similar upregulation of proangiogenic cytokines by culturing mouse neuroendocrine cell line under hypoxic condition in vitro. Similarly, in a human phase II study of sunitinib in LGNETs, VEGF receptor inhibition was associated with elevation of circulating VEGF concentration (47). These, together with our findings in the current study, suggest effective VEGF inhibition lead to hypoxic stress which if non-lethal may cause up-regulation of transcription factor Sp1. This stress response leads to broad activation of pro-angiogenic and survival pathways. This response, at least in part, may contribute to resistance of human LGNETs to therapy with VEGF inhibitors.

Combination therapy with BVZ and MIT blocks this resistance mechanism and represents a promising novel approach to enhancing the activity of anti-angiogenic therapy. Our findings not only provide researchers with a innovative paradigm of synergism between anti-angiogenesis agents and inhibitors of transcription factors such as Sp1, but also will help clinicians design rational combination drug therapy for improved anti-tumor activity.

Acknowledgments

We thank Cindi Tomlin Stokely for her expert help in the preparation of this manuscript, and Don Norwood for editorial comments.

### REFERENCES

1. Obendorfer S. Karzinoide Tumoren des Dunndarms. Frankf Z Pathol 1907; 1:425-9.
2. Modlin I M, Lye K D, Kidd M. A 5-decade analysis of 13, 715 carcinoid tumors. Cancer 2003 Feb. 15; 97(4):934-59.
3. Yao J C, Hassan M, Phan A, et al. One Hundred Years After “Carcinoid”: Epidemiology of and Prognostic Factors for Neuroendocrine Tumors in 35,825 Cases in the United States. J Clin Oncol 2008 (in press).
4. Kulke M H, Mayer R J. Carcinoid tumors. N Engl J Med 1999; 340(11):858-68.
5. Schnirer T I, Yao J C, Ajani J A. Carcinoid: A comprehensive review. Acta Oncol 2003; 42(7):672-92.
6. Lubensky I A, Pack S, Ault D, et al. Multiple neuroendocrine tumors of the pancreas in von Hippel-Lindau disease patients: histopathological and molecular genetic analysis. Am J Pathol 1998 July; 153(1):223-31.
7. Hammel P R, Vilgrain V, Terris B, et al. Pancreatic involvement in von Hippel-Lindau disease. The Groupe Francophone d'Etude de la Maladie de von Hippel-Lindau. Gastroenterology 2000 October; 119(4):1087-95.
8. Verhoef S, van Diemen-Steenvoorde R, Akkersdijk W L, et al. Malignant pancreatic tumour within the spectrum of tuberous sclerosis complex in childhood. Eur J Pediatr 1999 April; 158(4):284-7.
9. Johnson L, Weaver M. Von Recklinghausen's disease and gastrointestinal carcinoids. JAMA 1981; 245(24):2496.
10. Wang G G, Yao J C, Worah S, et al. Comparison of genetic alterations in neuroendocrine tumors: frequent loss of chromosome 18 in ileal carcinoid tumors. Mod Pathol 2005 August; 18(8):1079-87.
11. Hurr K, Kemp B, Silver S A, el-Naggar A K. Microsatellite alteration at chromosome 3p loci in neuroendocrine and non-neuroendocrine lung tumors. Histogenetic and clinical relevance. Am J Pathol 1996; 149(2):613-20.
12. Kovatich A, Friedland D M, Druck T, et al. Molecular alterations to human chromosome 3p loci in neuroendocrine lung tumors. Cancer 1998; 83(6):1109-17.
13. Li J H, Zhao H Z, Chen L A. [Nd-YAG laser treatment of tracheobronchial lesions]. Chung HuaNei Ko Tsa Chih 1989; 28(7):418-20, 44-5.
14. Chaudhry A, Funa K, Oberg K. Expression of growth factor peptides and their receptors in neuroendocrine tumors of the digestive system. Acta Oncol 1993; 32(2): 107-14.
15. Krishnamurthy S, Dayal Y. Immunohistochemical expression of transforming growth factor alpha and epidermal growth factor receptor in gastrointestinal carcinoids. Am J Surg Pathol 1997; 21(3):327-33.
16. Nilsson O, Wangberg B, Kolby L, Schultz G S, Ahlman H. Expression of transforming growth factor alpha and its receptor in human neuroendocrine tumours. Int J Cancer 1995; 60(5):645-51.
17. Papouchado B, Erickson L A, Rohlinger A L, et al. Epidermal growth factor receptor and activated epidermal growth factor receptor expression in gastrointestinal carcinoids and pancreatic endocrine carcinomas. Mod Pathol 2005 October; 18(10):1329-35.
18. von Wichert G, Jehle P M, Hoeflich A, et al. Insulin-like growth factor-I is an autocrine regulator of chromogranin A secretion and growth in human neuroendocrine tumor cells. Cancer Res 2000 Aug. 15; 60(16):4573-81.
19. Terris B, Scoazec J Y, Rubbia L, et al. Expression of vascular endothelial growth factor in digestive neuroendocrine tumours. Histopathology 1998; 32(2):133-8.
20. Yao J C, Wang L, Wei D, et al. Association between expression of transcription factor Sp1 and increased vascular endothelial growth factor expression, advanced stage, and poor survival in patients with resected gastric cancer. Clin Cancer Res 2004 Jun. 15; 10(12 Pt 1):4109-17.
21. Shi Q, Le X, Abbruzzese J L, et al. Constitutive Sp1 activity is essential for differential constitutive expression of vascular endothelial growth factor in human pancreatic adenocarcinoma. Cancer Research 2001; 61(10):4143-54.
22. Zhang J, Jia Z, Li Q, et al. Elevated expression of vascular endothelial growth factor correlates with increased angiogenesis and decreased progression-free survival among patients with low-grade neuroendocrine tumors. Cancer 2007 Mar. 5; 109(8):1478-86.
23. Black A R, Black J D, Azizkhan-Clifford J. Sp1 and kruppel-like factor family of transcription factors in cell growth regulation and cancer. J Cell Physiol 2001 August; 188(2): 143-60.
24. Li L, He S, Sun J M, Davie J R. Gene regulation by Sp1 and Sp3. Biochem Cell Biol 2004 August; 82(4):460-71.
25. Mukhopadhyay D, Datta K. Multiple regulatory pathways of vascular permeability factor/vascular endothelial growth factor (VPF/VEGF) expression in tumors. Semin Cancer Biol 2004 April; 14(2):123-30.
26. Abdelrahim M, Smith R, 3rd, Burghardt R, Safe S. Role of Sp proteins in regulation of vascular endothelial growth factor expression and proliferation of pancreatic cancer cells. Cancer Res 2004 Sep. 15; 64(18):6740-9.
27. Perlman J A, Hoover H C, Safer P K. Femoral hernia with strangulated Meckel's diverticulum (Littre's hernia). Am J Surg 1980; 139(2):286-9.
28. Wang L, Wei D, Huang S, et al. Transcription factor Sp1 expression is a significant predictor of survival in human gastric cancer. Clin Cancer Res 2003 Dec. 15; 9(17):6371-80.
29. Wohlert S E, Kunzel E, Machinek R, Mendez C, Salas J A, Rohr J. The structure of mithramycin reinvestigated. J Nat Prod 1999 January; 62(1): 119-21.
30. Prado L, Lombo F, Brana A F, Mendez C, Rohr J, Salas J A. Analysis of two chromosomal regions adjacent to genes for a type II polyketide synthase involved in the biosynthesis of the antitumor polyketide mithramycin in Streptomyces argillaceus. Mol Gen Genet. 1999 March; 261(2):216-25.
31. Majee S, Dasgupta D, Chakrabarti A. Interaction of the DNA-binding antitumor antibiotics, chromomycin and mithramycin with erythroid spectrin. Eur J Biochem 1999 March; 260(3):619-26.
32. Hall T J, Schaeublin M, Chambers T J. The majority of osteoclasts require mRNA and protein synthesis for bone resorption in vitro. Biochem Biophys Res Commun 1993 Sep. 30; 195(3):1245-53.
33. Remsing L L, Bahadori H R, Carbone G M, McGuffie E M, Catapano C V, Rohr J. Inhibition of c-src transcription by mithramycin: structure-activity relationships of biosynthetically produced mithramycin analogues using the c-src promoter as target. Biochemistry 2003 Jul. 15; 42(27):8313-24.
34. Blume S W, Snyder R C, Ray R, Thomas S, Koller C A, Miller D M. Mithramycin inhibits SP1 binding and selectively inhibits transcriptional activity of the dihydrofolate reductase gene in vitro and in vivo. J Clin Invest 1991 November; 88(5):1613-21.
35. Tagashira M, Kitagawa T, Isonishi S, Okamoto A, Ochiai K, Ohtake Y. Mithramycin represses MDR1 gene expression in vitro, modulating multidrug resistance. Biol Pharm Bull 2000 August; 23(8):926-9.
36. Duverger V, Murphy A M, Sheehan D, et al. The anticancer drug mithramycin A sensitises tumour cells to apoptosis induced by tumour necrosis factor (TNF). Br J Cancer 2004 May 17; 90(10):2025-31.
37. Koutsodontis G, Kardassis D. Inhibition of p53-mediated transcriptional responses by mithramycin A. Oncogene 2004 10 18.
38. Evers B M, Townsend C M, Jr., Upp J R, et al. Establishment and characterization of a human carcinoid in nude mice and effect of various agents on tumor growth. Gastroenterology 1991 August; 101(2):303-11.
39. Kanai M, Wei D, Li Q, et al. Loss of Kruppel-like factor 4 expression contributes to Sp1 overexpression and human gastric cancer development and progression. Clin Cancer Res 2006 Nov. 1; 12(21):6395-402.
40. Weidner F A, Ziter F M, Jr. Carcinoid tumors of the gastrointestinal tract. Jama 1981; 245(11):1153-5.
41. Ambs S, Bennett W P, Merriam W G, et al. Vascular endothelial growth factor and nitric oxide synthase expression in human lung cancer and the relation to p53. Br J Cancer 1998 July; 78(2):233-9.
42. Hobday T J, Rubin J, Goldberg R, Erlichman C, Lloyd R. Molecular markers in metastatic gastrointestinal neuroendocrine tumors. Annual Meeting of American Society of Clinical Oncology; 2003 May 31, 2003; Chicago, Ill.: American Society of Clinical Oncology; 2003. p. 269.
43. Couvelard A, O'Toole D, Turley H, et al. Microvascular density and hypoxia-inducible factor pathway in pancreatic endocrine tumours: negative correlation of microvascular density and VEGF expression with tumour progression. Br J Cancer 2005 Jan. 17; 92(1):94-101.
44. Kulke M H, Lenz H J, Meropol N J, et al. A phase 2 study to evaluate the efficacy and safety of SU 11248 in patients (pts) with unresectable neuroendocrine tumors (NETs). J Clin Oncol 2005; 23(16s):310s.
45. Hobday T J, Rubin J, Holen K, et al. M C044h, a phase II trial of sorafenib in patients (pts) with metastatic neuroendocrine tumors (NET): A Phase II Consortium (P2C) study. J Clin Oncol 2007; 25(18S):4504.
46. Duran I, Salazar R, Casanovas O, et al. New drug development in digestive neuroendocrine tumors. Ann Oncol 2007 Feb. 13.
47. Bello C L, Deprimo S E, Friece C, et al. Analysis of circulating biomarkers of sunitinib malate in patients with unresectable neuroendocrine tumors (NET): VEGF, IL-8, and soluble VEGF receptors 2 and 3. J Clin Oncol 2006; 24(18s):189s.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps thereof described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Yao J C, Wang L, Wei D, Gong W, Hassan M, Wu T T, et al. Association between expression of transcription factor Sp1 and increased vascular endothelial growth factor expression, advanced stage, and poor survival in patients with resected gastric cancer. Clin Cancer Res 2004; 10(12 Pt 1):4109-17.
2. von Marschall Z, Scholz A, Cramer T, Schafer G, Schirner M, Oberg K, et al. Effects of interferon alpha on vascular endothelial growth factor gene transcription and tumor angiogenesis. [comment]. Journal of the National Cancer Institute 2003; 95(6):437-48.
3. Shi Q, Le X, Abbruzzese J L, Peng Z, Qian C N, Tang H, et al. Constitutive Sp1 activity is essential for differential constitutive expression of vascular endothelial growth factor in human pancreatic adenocarcinoma. Cancer Research 2001; 61(10):4143-54.
4. Finkenzeller G, Sparacio A, Technau A, Marme D, Siemeister G. Sp1 recognition sites in the proximal promoter of the human vascular endothelial growth factor gene are essential for platelet-derived growth factor-induced gene expression. Oncogene 1997; 15(6):669-76.
5. Luster T A, Johnson L R, Nowling T K, Lamb K A, Philipsen S, Rizzino A. Effects of three Sp1 motifs on the transcription of the FGF-4 gene. Mol Reprod Dev 2000; 57(1):415.
6. Blume S W, Snyder R C, Ray R, Thomas S, Koller C A, Miller D M. Mithramycin inhibits SP1 binding and selectively inhibits transcriptional activity of the dihydrofolate reductase gene in vitro and in vivo. J Clin Invest 1991; 88(5):1613-21.
7. Evers B M, Townsend C M, Jr., Upp J R, Allen E, Hurlbut S C, Kim S W, et al. Establishment and characterization of a human carcinoid in nude mice and effect of various agents on tumor growth. Gastroenterology 1991; 101 (2):303-11.
8. Nishikawa S, Rosol T J, Capen C C. Effects of mithramycin on transplantable canine perianal gland carcinoma (CAC-9) in nude mice: biochemical, histomorphometric, and ultrastructural investigations. Vet Pathol 1986; 23(6):698-705.
9. Rosol T J, Capen C C. The effect of low calcium diet, mithramycin, and dichlorodimethylene bisphosphonate on humoral hypercalcemia of malignancy in nude mice transplanted with the canine adenocarcinoma tumor line (CAC-8). J Bone Miner Res 1987; 2(5):395-405.)]

What is claimed is:

1. A method of inhibiting angiogenesis in a mammal, comprising:
administering by a subcutaneous or intraperitoneal route to a tissue in the mammal where angiogenesis is occurring a composition comprising mithramycin and a pharmaceutically-acceptable carrier.

2. The method of claim 1, wherein the mammal is Homo sapiens.

3. The method of claim 2, wherein the mammal suffers from a medical condition selected from the group consisting of cancer, macular degeneration, diabetic neuropathy, psoriasis, and rheumatoid arthritis.

4. The method of claim 3, wherein the cancer is selected from the group consisting of gastric cancer, carcinoid cancer, pancreatic cancer, and ovarian cancer.

5. The method of claim 1, wherein administering delivers a dose from about 10 μg mithramycin per kg body weight per day to about 500 μg mithramycin per kg body weight per day.

6. The method of claim 1, wherein the composition further comprises a VEGF antagonist or a PDGF antagonist.

7. The method of claim 6, wherein the VEGF antagonist is bevcaizumab or the PDGF antagonist is imatinib.

8. The method of claim 6, wherein administering delivers a dose from about 100 μg bevcaizumab per kg body weight per day to about 4 mg bevcaizumab per kg body weight per day.

9. The method of claim 1, wherein the pharmaceutically-acceptable carrier is selected from the group consisting of distilled water, normal saline, and phosphate-buffered saline (PBS).
